(19) United States
(12) Patent Application Publication
(10) Pub. No.: US 2020/0202997 A1
(43) Pub. Date: Jun. 25, 2020

(54) AUTOMATED METHOD AND SYSTEM FOR GENERATING PERSONALIZED DIETARY AND HEALTH ADVICE OR RECOMMENDATIONS FOR INDIVIDUAL USERS

(71) Applicant: NUTRINO HEALTH LTD., Tel-Aviv (IL)

(72) Inventors: Yaron Hadad, Northridge, CA (US); Daniel Modlinger, Northridge, CA (US)

(21) Appl. No.: 16/709,721

(22) Filed: Dec. 10, 2019

Related U.S. Application Data
(60) Provisional application No. 62/782,275, filed on Dec. 19, 2018.

(51) Int. Cl. G16H 20/60; H04L 9/32; G16H 50/20; G06N 20/00; G16H 50/30; G06F 21/62; G16H 50/70

(52) U.S. Cl. CPC G16H 20/60; H04L 9/3213; G16H 50/20; G16H 50/70; G16H 50/30; G06F 21/6245; G06N 20/00

(57) ABSTRACT

A method, system and platform are provided that can utilize a serverless architecture with autonomous functions to standardize nutritional and health data from various sources into a structured file format suitable for analysis. The platform may include an authentication component, a data retrieving component, a pipeline component, a standardization component, and a storage component. The components may include sets of autonomous functions, streaming applications, notification messages, and other objects logically connected to one another. The components may be connected serially and data may flow through the components sequentially in a stream. Using the disclosed architecture, the platform can aggregate and process large volumes of data in an efficient and cost-effective manner, analyze the standardized structured data, and generate personalized dietary and health advice or recommendations to individual end users.

### CROSS-REFERENCE TO RELATED APPLICATIONS

This application claims priority to U.S. Prov. Pat. App. No. 62/782,275, filed Dec. 19, 2018, the contents of which are hereby incorporated by reference in their entirety.

### TECHNICAL FIELD

Embodiments of the subject matter described herein relate generally to providing personalized dietary and health advice or recommendations, and techniques and technologies for automatically creating the same. More particularly, embodiments of the subject matter relate to a serverless architecture that automatically collects and processes data to generate personalized dietary and health advice or recommendations for individual users.

### BACKGROUND

In recent years, a number of devices and software applications have been developed to deliver health-related data to customers. These devices and software applications can monitor activities, allow people to monitor their food consumption and exercise habits, monitor sleep patterns, and passively collect health information from users. However, there are presently no industry standards for standardizing and collectively processing all this data. In particular, the data collected is difficult to consolidate since the data is obtained from various sources in a variety of different formats. This makes it difficult for users to have complete information about their nutritional needs, and thus inhibits the ability of users to make timely and informed decisions about food consumption and impact of different foods on their health.

Consolidating and processing various food-related, nutritional and health data poses many challenges. For example, the data may be provided as different data types (e.g. structured or unstructured, time-series, etc.), and processed using different methods or tools in order to extract and convey useful information. In addition, the amount of data collected with these devices and software applications can be massive, on the order of thousands or millions of data points being collected on a frequent basis at regular or random intervals. The amount of data to be collected tends to exponentially increase over time as devices and software applications become increasingly interconnected with the users' everyday lives. In some cases, when updates are made to application programming interfaces (APIs), the changes may cause certain APIs to malfunction resulting in data loss.

Accordingly, it is desirable to provide technologies, systems, methods and techniques for addressing such issues including challenges relating to the consolidation and processing of food-related, nutritional and health data from various disparate sources. Furthermore, other desirable features and characteristics will become apparent from the subsequent detailed description and the appended claims, taken in conjunction with the accompanying drawings and the foregoing technical field and background.

### BRIEF SUMMARY

A platform is disclosed herein that can handle large amounts of food-related, nutritional and health data in a scalable manner, and manage unpredictability in the rate at which data is generated and processed. The disclosed platform is also capable of handling many different data types and can be adapted to receive data from multiple different sources. The platform can also support changes made to the underlying APIs, without experiencing issues relating to data processing or data loss.

The platform disclosed herein may include one or more modules that allow for processing, consolidation, and structuring of large amounts of food-related, nutritional and health data. The modules may be decoupled from one another, thereby ensuring ease of maintenance and reusability of each component or module. The platform disclosed herein can handle large volumes of data by employing a serverless architecture. In a serverless architecture, leveraging tools such as AMAZON® Lambda, data may be streamed through the platform, and code processing may be performed only when processing functions are triggered by the streaming data. Such functions are generally known as “lambda functions”. Using lambda functions in this manner can allow computing resources for processing data to be used more efficiently, because it does not require computing resources to be continuously running. The serverless architecture can enable large volumes of data to be efficiently processed by the platform disclosed herein.

The platform components can be configured to interface with many data types. In a retrieving module, a set of lambda functions may be configured to pull data from connected applications, and implement a time-based task scheduler that retrieves data on a periodic basis. Another set of lambda functions may receive notifications from connected applications and prepare to receive pushed data. Another set of lambda functions may consolidate the pulled and pushed data, and send the data to a stream that cascades through the platform. Additional lambda functions may divert the data from the stream for processing, which may convert the data into a standardized, structured format. The converted structured data can be further analyzed in order to provide users with insights or recommendations on nutrition and health.

In one embodiment of the disclosure, a data collection and processing method is provided. The method may be implemented using a serverless architecture. The serverless architecture can enable the method to be scaled, and to support new types or forms of data, or new sources as they are introduced. The method disclosed herein may include collecting and aggregating data from a plurality of different sources, wherein the data comprises different types or forms of data. The different types or forms of data may include structured data and unstructured data, as well as time-series sensor data. The data may include food, health or nutritional data that is specific to a plurality of individual users. The method may further include continuously processing each of the different types or forms of data in a manner that is agnostic of its source, by converting the different types or forms of data to a standardized structured format that is compatible with a health and nutrition platform. The method may also include analyzing the data that has been converted to the standardized structured format, using in part information from the health and nutrition platform. The standardized structured data may be analyzed using one or more machine learning models. Based on the analysis, personalized dietary and health advice or recommendations can be generated for each of a plurality of individual users.

In some embodiments, the plurality of different sources may include two or more of the following: mobile devices, wearable devices, medical devices, home appliances, or healthcare databases. The mobile devices may include smart devices (e.g., smartphones, tablets), and wherein the wearable devices comprise one or more of the following: activity trackers, smartwatches, smart glasses, smart rings, smart patches, antioxidant monitors, sleep sensors, biomarker blood monitors, heart rate variability (HRV) monitors, stress monitors, temperature monitors, automatic scales, fat monitors, or smart fabrics. The medical devices may include one or more of the following: glucose monitors, heart rate monitors, blood pressure monitors, sweat sensors, insulin pumps, ketone monitors, lactic acid monitors, iron monitors, or galvanic skin response (GSR) sensors. Exemplary embodiments of the subject matter described herein can be implemented in conjunction with medical devices, such as portable electronic medical devices. Although many different applications are possible, one embodiment can incorporate an insulin infusion device (or insulin pump) as part of an infusion system deployment. For the sake of brevity, conventional techniques related to infusion system operation, insulin pump and/or infusion set operation, and other functional aspects of the systems (and the individual operating components of the systems) may not be described in detail here. Examples of infusion pumps (e.g., insulin pumps) may be of the type described in, but not limited to, U.S. Pat. Nos. 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893; each of which are herein incorporated by reference. The healthcare databases may include genetic databases, blood test database, biome databases, or electronic medical records (EMR).

In some embodiments, the data from the plurality of different sources may include at least on the order of 106 daily data points that are unevenly distributed throughout a day. The data may be collected and aggregated from the plurality of different sources through a plurality of Application Programming Interfaces (APIs). In some cases, the processing of the data is not subject to changes or updates to the underlying APIs, such that the data is capable of being processed without loss of data as changes or updates are made to the underlying APIs.

In some embodiments, the collecting and aggregating of the data may include storing the data in a plurality of streams. The processing of the data may further include executing lambda functions on the data stored in the plurality of streams, upon occurrence of different conditions. The lambda functions may be executed only when the data is collected and stored in the plurality of streams. The executing of the lambda functions on the stored data is configured to channel and transfer each row of data to a relevant stream from the plurality of streams. The data can be advanced along a data pipeline by cascading from one stream to another stream of the plurality of streams.

In some embodiments, the collecting and aggregating of the data from the plurality of different sources may include (1) pulling data from a first set of sources that permit data to be pulled, and (2) receiving data that is pushed from a second set of sources, such that the data from a plurality of pull requests and push requests are streamed into a centralized location. The data may be pulled from the first set of sources at predetermined time intervals using a task scheduler. The data may also be received from the second set of sources, as or when the data is pushed from the second set of sources. In some cases, the pushing of the data from the second set of sources may be preceded by one or more notifications associated with the data. In other cases, the data associated with a corresponding notification if the data does not arrive with the corresponding notification. In some instances, the first set of sources and the second set of sources may include one or more sources that are common to both the first and second sets. In other instances, the first set of sources and the second set of sources may include sources that are different from one another.

In some embodiments, each of the plurality of streams may have a retention policy defining a time frame in which the data is stored in each stream. The time frame may range, for example from about 24 hours to about 168 hours. The data can be stored in the plurality of streams in a decoupled manner without requiring prior knowledge of the sources(s) of each data. The plurality of streams may include a plurality of shards. Each shard may include a string of data records that (1) enter into a queue and (2) exit the queue upon expiration of the retention policy. The string of data records may include food consumption, health or nutritional records that are specific to a plurality of individual users. A speed at which the data is being processed can be controlled, by controlling a number of shards in the plurality of streams.

In some embodiments, the method may include communicating with the plurality of APIs via a token module associated with one or more different entities. The data from the plurality of APIs may be collected and aggregated using a retrieving module, whereby the retrieving module may be decoupled from and independent of the token module. The token module may be configured to refresh existing tokens and provide notification updates about token changes. Each time a new token is generated, the new token may be separately duplicated in the retrieving module, in addition to being stored in the token module. In some cases, the retrieving module may be only configured to collect and aggregate the data, and is not configured to persist, store, or process the data.

In some embodiments, some or all of the collected data may be provided to and utilized in a health and nutrition platform. Additionally or optionally, a portion of the collected data may be transmitted to one or more third parties. The data may be converted to the standardized structured format before it is provided to and utilized in the health and nutrition platform.

In some embodiments, the data from the plurality of data sources may be collected and aggregated in a storage module. The storage module may be configured to verify, check and remove duplicate data. The storage module may be configured to persist the data in batches. The storage module may be configured to reduce the data by consolidating selected types of data.

In some cases, a portion of the stored data may include a plurality of images captured using one or more imaging devices. A selected lambda function may be executed on the portion of the stored data, to detect whether any of the plurality of images comprises one or more food images that are to be analyzed for their nutritional contents. The one or more food images may be associated with timestamps and geolocations, thereby enabling temporal and spatial tracking of a user's food intake. The temporal and spatial tracking of the user's food intake may include predicting a time of consumption of a meal or a content of a meal.

In another embodiment of the disclosure, a serverless data collection and processing system is provided. The system may include a retrieving module configured to collect and aggregate data from a plurality of different sources, wherein the data comprises different types or forms of data. The system may also include a standardization module configured to continuously process each of the different types or forms of data in a manner that is agnostic of its source, by converting the different types or forms of data to a standardized structured format that is compatible with a health and nutrition platform.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter. It shall be understood that different embodiments of the disclosure can be appreciated individually, collectively, or in combination with each other. Various embodiments of the disclosure described herein may be applied to any of the particular applications set forth below or for any other types of health, nutrition or food-related monitoring/tracking/recommendation systems and methods.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 illustrates an ecosystem in accordance with some embodiments;

FIG. 2 shows a block diagram of a platform in accordance with some embodiments;

FIG. 3 shows the components of a token module in accordance with some embodiments;

FIG. 4 shows the components of a receiving module in accordance with some embodiments;

FIG. 5 shows the components of a pipeline module in accordance with some embodiments;

FIG. 6 shows the components of a standardization module in accordance with some embodiments;

FIG. 7 shows the components of a storage module in accordance with some embodiments;

FIG. 8 shows an example of the token module of FIG. 3 in accordance with some embodiments;

FIG. 9 shows an example of the retrieving module of FIG. 4 in accordance with some embodiments;

FIG. 10 shows an example of the pipeline module of FIG. 5 in accordance with some embodiments;

FIG. 11 shows an example of the standardization module of FIG. 6 in accordance with some embodiments;

FIG. 12 shows an example of the storage module of FIG. 7 in accordance with some embodiments;

FIG. 13 is a flowchart that illustrates a computer-implemented data collection and processing method that is implemented using a serverless architecture including a health and nutrition platform for generating personalized dietary and health advice or recommendations via a hardware-based processing system in accordance with the disclosed embodiments;

FIG. 14 is a flowchart that illustrates a method for collecting and aggregating of the data from the plurality of different sources in accordance with the disclosed embodiments;

FIG. 15 is a flowchart that illustrates a method for collecting and aggregating data from a plurality of different sources in a storage module in accordance with the disclosed embodiments;

FIG. 16 is a flowchart that illustrates a method for storing collected and aggregated data from a plurality of different sources and processing the collected and aggregated data in a storage module accordance with the disclosed embodiments;

FIG. 17 is a flowchart that illustrates a method for storing collected and aggregated data from a plurality of different sources in a plurality of streams in accordance with the disclosed embodiments; and

FIG. 18 is a flowchart that illustrates a method for analyzing images to determine their nutritional contents in accordance with the disclosed embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description. In addition, it should be noted that all publications, patents, and patent applications mentioned in this specification are herein incorporated by reference to the same extent as if each individual publication, patent, or patent application was specifically and individually indicated to be incorporated by reference.

Reference will now be made in detail to exemplary embodiments of the disclosure, examples of which are illustrated in the accompanying drawings. Wherever possible, the same reference numbers will be used throughout the drawings and disclosure to refer to the same or like parts.

Many applications exist today that collect health, nutrition, and fitness data from individuals. Some applications may be implemented on mobile phones or wearable devices, and can passively track activity levels and vital statistics, such as heartbeat, blood pressure, and insulin levels. Some applications may allow users to log their diets, exercise routines, and sleep habits, and may calculate health metrics from the logged data. It can be difficult for individuals to keep track of the myriad data obtained from multiple applications. Discrete data sets often may not provide the necessary insights to users, especially if the users do not fully comprehend the impact and relations between different types or sets of health and nutrition data. As a result, users may lack the necessary tools to take actionable steps to improve their health or well-being. A platform (as disclosed herein) can be configured to collect and consolidate vast amounts of health, food-related and nutrition data from multiple applications, and process the data in order to provide users with more precise and useful nutrition and/or health information. In some instances, neural networks and other machine learning algorithms can be used to analyze the data and provide individual users with personalized health recommendations.

The platform disclosed herein can (1) collect and aggregate data submitted by users and/or retrieved from different types of third-party applications, and (2) process the data in a manner that is agnostic of its source, by converting different types or forms of data to a standardized structured format that is compatible with a health and nutrition platform. In some embodiments, the platform disclosed herein may be integrated with, or provided as part of a health and nutrition platform. In other embodiments, the platform disclosed herein may be provided separate from a health and nutrition platform. Any modifications to the platform disclosed herein, or to a health and nutrition platform consistent with the present disclosure may be contemplated. Examples of health and nutrition platforms are described in U.S. patent application Ser. Nos. 13/784,845 and 15/981,832, both of which are incorporated herein by reference in their entirety.

The platform disclosed herein can be implemented using a serverless architecture with autonomous functions to process large volumes of health and nutritional data as the data is being streamed through the platform. Employing a serverless architecture can permit the platform to process large volumes of data, for example on the order of 106 data points per day, which may be distributed evenly or unevenly throughout a day. Using a serverless architecture is advantageous in mitigating the unpredictability associated with large fluctuations in data traffic, since server resources are utilized as and when needed, depending on incoming data flow. Autonomous functions can be triggered in response to specific events, such as when data items are received or stored. Implementing the platform using a serverless architecture can also provide cost benefits, since charges may be incurred only when certain functions are called. Furthermore, the functions may run for a short period of time, which eliminates the costs associated with continuous processor usage. When data is not being received, the autonomous functions need not be triggered, and thus processing costs are not incurred. Additional advantages of implementing the platform with a serverless architecture is that such architecture can permit scalability without incurring the costs associated using conventional server-based systems. As more data is processed by the serverless platform, the number of calls that trigger autonomous functions for processing the data will increase. The additional costs are based on the increased number of function calls. Savings can be realized using the disclosed platform since investments in additional server resources, maintenance, or personnel can be obviated using a serverless architecture.

A serverless architecture as described herein can be a software design deployment where applications are hosted by a third party services. Examples of third party services may include AMAZON® Web Services Lambda, TWILIO® Functions, and MICROSOFT® Azure Functions. Typically, hosting a server application on the Internet requires managing a virtual or physical server, as well as the operating system and other web server hosting processes required to run the application. Hosting applications on third-party services in a serverless architecture transfers the burdens of server software and hardware management to the third-party services.

Applications developed to work within a serverless architecture can be broken up by individual autonomous functions, which can be invoked and scaled individually. In the example of some third-party services described herein, the functions may be known for example as Lambda Functions, Twilio Functions, and Azure Functions. These functions are stateless containers that perform computing operations when they are triggered in response to events. They are ephemeral, which means that they may use computing power during one invocation or for a time period containing a limited number of invocations, instead of continuously using computing power. The autonomous functions may be fully managed by the third-party service. Serverless architectures with autonomous functions may be sometimes referred to as “Functions as a Service (FaaS).” Autonomous functions may be implemented using a variety of programming languages, depending on which languages are supported by the underlying serverless architecture. Example languages include JavaScript, Python, Go, Java, C, and Scala.

Computing tasks performed by autonomous functions may include storing data, triggering notifications, processing files, scheduling tasks, and extending applications. For example, an autonomous function may receive a request as an application programming interface (API) call from a mobile application, inspect values belonging to parameters within the request, perform an operation based on the inspected values, producing an output, and store the output data in a database by modifying table entries within the database. An example of processing operation performed by autonomous function may be optical character recognition (OCR) on PDF files or image files, converting symbols into editable text. Examples of scheduled tasks may be periodically removing duplicate entries from a database, requesting data from connected applications, and renewing access tokens. Autonomous functions may act as extensions of applications, retrieving data from the applications and posting the data to third party services for processing. For example, a service desk ticket may be forwarded, using an autonomous function, to a separate help desk chat program to be seen by staff.

An advantage of using serverless architectures such as those described herein is that they are easily scalable. Horizontal scaling, or adding additional resources, can be performed as resources are needed. For example, if the amount of processed requests expands, the architecture can automatically procure additional computing resources. The ephemeral autonomous functions can make scaling easier because they can be created and destroyed according to runtime need. Because the serverless architecture is standardized, it is easier to maintain if/when issues occur.

Another advantage of using serverless architectures is that serverless architectures can be cost-effective. Because autonomous functions are ephemeral, computing power may only be used when a function is invoked. Thus, when a function is not being invoked, there is no charge for computing power. This pay structure has advantages when requests are only occasional, or when traffic is inconsistent. If a server is being run continuously, but only processes one request per minute, the server may be inefficient because the amount of time processing the request is low compared to the time the server is up and running. By contrast, with a serverless architecture, an ephemeral autonomous function would use computing power to handle the request and remain dormant the rest of the time. When traffic is inconsistent, little computing power may be used when requests are infrequent. When traffic spikes, a large amount of computing power may be used. In a traditional environment, a hardware count may need to increase to handle the traffic spikes, but the hardware would be wasted when traffic dies down. However, in a serverless environment, flexible scaling allows for increased payment only during traffic spikes, and money savings during low-traffic periods.

The serverless architecture disclosed herein can consolidate and process streaming data. Streaming data is data that is generated continuously by multiple sources and processed simultaneously. The serverless architecture can collect and process the streaming data quickly and in a timely manner (e.g. substantially in real time), as the data is generated. This contrasts with gathering data, storing it in a database, and analyzing it later. The serverless architecture may have services specially designed to capture, transform, and analyze the data. These services may complement autonomous functions to compress, encrypt, and convert streaming data into formats that are interoperable with different kinds of third-party applications.

The autonomous functions can enable the platform to perform many tasks, for example authentication, authorization, data consolidation, data transportation, data processing, and standardization, etc. Certain autonomous functions can communicate with external application programming interfaces (APIs) and exchange, store, renew, and delete access tokens to manage application permissions. Some of the autonomous functions can retrieve data from connected applications that push and pull data into the platform, and consolidate all of the collected data into streams. Other autonomous functions can transport the streamed data to other components of the platform. Some other autonomous functions can process the data by sorting the data, converting the data into different file formats, removing redundant data, and/or standardizing the data. Some other autonomous functions can pre-process the data for storage and analysis.

The modules in the platform may be decoupled to permit ease of maintenance or updates. A module as described herein may be referred to interchangeably as a component. Conversely, a module as described herein may include one or more components, such that the module comprises a group of components. By decoupling the modules, data can flow through the platform components and can be processed without data loss. For example, tokens can be copied from one component to another component, and the two components may be decoupled such that they do not depend on each other. In some cases, one component may be configured for redirecting the stream, while another component may be configured for processing. A third component may be configured for storage. The platform disclosed herein can be designed in a modular fashion, with each module configured to perform a specific function without requiring inter-operational dependency on one or more other modules.

The disclosed platform having a serverless architecture is well-suited for big data processing, providing the platform the flexibility to aggregate many different types or forms of data into a standardized structured format that is compatible with a health and nutrition platform, or with other third-party applications. The platform can collect data from users, and can also integrate with multiple APIs from various third-party applications to collect other types of data. In some embodiments, the platform can create a food ontology that is continuously being updated from various sources (e.g., from the Internet, pre-existing databases, user input, etc.) to organize and analyze any obtainable information of all food types (e.g., elementary foods, packaged foods, recipes, restaurant dishes, etc.). In some embodiments, the platform can also enable users to log information about the meals consumed, exercise or activities performed, amount of sleep, and other health data manually. In some embodiments, the platform's integrations with third party applications can allow the platform to generate a personalized data network among a multitude of data collection devices and services (e.g., mobile devices, glucose sensors, healthcare provider databases, etc.) to integrate any obtainable information of biomarkers that can be affected by, or that can affect metabolism (e.g., sleep, exercise, blood tests, stress, blood sugar, DNA etc.). Integration of the platform with medical devices manufactured by companies such as Medtronic, Abbott, Dexcom, etc. can provide the platform with data, such as device usage data and health-related data. The platform can synthesize the food ontology, manual logs, and personalized data network by connecting or correlating the various information to draw insights on how different foods can affect each individual, and further generate personalized food, health, and wellness recommendations for each individual.

Embodiments of the platform may utilize for example AMAZON® Web Service solutions, including AMAZON® Lambda, AMAZON® S3, and AMAZON® Kinesis. Other embodiments may utilize analogous tools from services such as GOOGLE® Cloud Services or MICROSOFT® Azure.

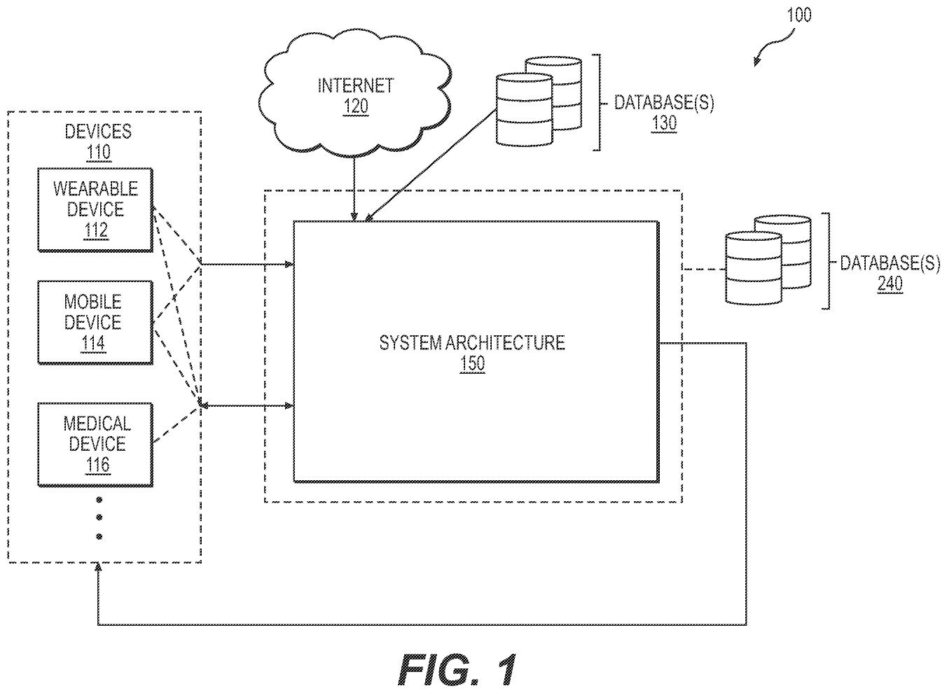

The following description with reference to the figures provide context to the environment in which the platform can be implemented, and describes in detail the structure of the platform as well as data streams through the platform. FIG. 1 illustrates an ecosystem 100 in accordance with some embodiments. In one aspect, the ecosystem 100 can include system architecture or platform 150. The platform may collect and aggregate data from a plurality of different sources (e.g. devices 110, Internet 120, and database(s) 130). As shown in FIG. 1, the ecosystem 100 can include devices 110. The devices 110 can include a wearable device 112 (e.g., a smart watch, activity tracker, smart glasses, smart rings, smart patches, smart fabrics, etc.), a mobile device 114 (e.g., a cell phone, a smart phone, a voice recorder, etc.), and/or a medical device 116 (e.g. a glucose monitor, insulin pump, blood pressure monitor, heart rate monitor, sweat sensor, galvanic skin response (GSR) monitor, skin temperature sensor, etc.). In some instances, the devices 110 may include home appliances (e.g., smart refrigerators that can track food and diet habits, smart microwaves that can track the amount and type of food being consumed, etc.) or game consoles that can track user physical activity level. The devices 110 can be in communication with one another. The platform 150 can be in communication with one or more of the devices 110, either concurrently or at different time instances.

The devices 110 may comprise one or more sensors. The sensors can be any device, module, unit, or subsystem that is configured to detect a signal or acquire information. Non-limiting examples of sensors may include inertial sensors (e.g., accelerometer, gyroscopes, gravity detection sensors which may form inertial measurement units (IMUs)), location sensors (e.g., global positioning system (GPS) sensors, mobile device transmitters enabling location triangulation), heart rate monitors, temperature sensors (e.g., external temperature sensors, skin temperature sensors), environmental sensors configured to detect parameters associated with an environment surrounding the user (e.g., temperature, humidity, brightness), capacitive touch sensors, GSR sensors, vision sensors (e.g., imaging devices capable of detecting visible, infrared, or ultraviolet light, cameras), thermal imaging sensors, location sensors, proximity of range sensors (e.g., ultrasound sensors, light detection and ranging (LIDAR), time-of-flight or depth cameras), altitude sensors, attitude sensors (e.g., compasses), pressure sensors (e.g., barometers), humidity sensors, vibration sensors, audio sensors (e.g., microphones), field sensors (e.g., magnetometers, electromagnetic sensors, radio sensors), sensors used in HRV monitors (e.g., electrocardiogram (ECG) sensors, ballistocardiogram sensors, photoplethysmogram (PPG) sensors), blood pressure sensors, liquid detectors, Wi-Fi, Bluetooth, cellular network signal strength detectors, ambient light sensors, ultraviolet (UV) sensors, oxygen saturation sensors, or combinations thereof or any other sensors or sensing devices, as described elsewhere herein. The sensors may be located on one or more of the wearable devices, mobile devices, or medical devices. In some cases, a sensor may be placed inside the body of a user.

The devices 110 may also include any computing device that can be in communication with platform 150. Non-limiting examples of computing devices may include mobile devices, smartphones/cellphones, tablets, personal digital assistants (PDAs), laptop or notebook computers, desktop computers, media content players, television sets, video gaming station/system, virtual reality systems, augmented reality systems, microphones, or any electronic device capable of analyzing, receiving, providing or displaying various types of health, nutrition or food data. A device may be a handheld object. A device may be portable. A device may be carried by a human user. In some cases, a device may be located remotely from a human user, and a user can control the device using wireless and/or wired communications.

The platform 150 can be in communication with the Internet 120 and database(s) 130 (e.g., other food, nutrition, or healthcare providers). For example, the platform may be in communication with healthcare databases containing electronic medical records (EMR). In some embodiments, the database(s) 130 may include data stored in an unstructured database or format, such as a Hadoop distributed file system (HDFS). A HDFS data store may provide storage for unstructured data. HDFS is a Java-based file system that provides scalable and reliable data storage, and can be designed to span large clusters of commodity servers. A HDFS data store may be beneficial for parallel processing algorithms such as MapReduce.

The platform 150 can also be in communication with additional database(s) 240 to store any data or information that is collected or generated by the platform 150. The additional database(s) 240 may be a collection of secure cloud databases. The data from the plurality of different sources may comprise different types or forms of data (structured data and/or unstructured data). In some cases, the data may include time-series data collected by one or more devices 110, sensors, or monitoring systems. The time-series data may include periodic sensor readings or other data. The platform may receive data from any number or type of devices, ranging from tens, hundreds, thousands, hundreds of thousands, or millions of devices. The platform 150 can continuously process each of the different types or forms of data in a manner that is agnostic of its source, by converting the different types or forms of data to a standardized structured format. The converted data in the standardized structured format may be compatible with a health and nutrition platform. As described elsewhere herein, the platform 150 may be integrated with, or provided as part of the health and nutrition platform. In some embodiments, the platform 150 may be provided separate from the health and nutrition platform.

The platform 150 may include a set of components (or modules) that can transfer streaming data from and between one another. In some embodiments, the data may be stored in persistent queues using AMAZON® Kinesis data streams. In some embodiments, the data may comprise food, health, or nutritional data that is specific to one or more individual users. The platform 150 can analyze the data that has been converted to the standardized structured format, using in part information from the health and nutrition platform. In some embodiments, the platform 150 can analyze the standardized structured data using one or more machine learning models or natural language processing (NLP) techniques. Machine learning models or algorithms that may be used in the present disclosure may comprise supervised (or predictive) learning, semi-supervised learning, active learning, unsupervised machine learning, or reinforcement learning.

Artificial intelligence is an area of computer science emphasizes the creation of intelligent machines that work and react like humans. Some of the activities computers with artificial intelligence are designed for include learning. Examples of artificial intelligence algorithms include, but are not limited to, key learning, actor critic methods, reinforce, deep deterministic policy gradient (DDPG), multi-agent deep deterministic policy gradient (MADDPG), etc. Machine learning refers to an artificial intelligence discipline geared toward the technological development of human knowledge.

Machine learning facilitates a continuous advancement of computing through exposure to new scenarios, testing and adaptation, while employing pattern and trend detection for improved decisions and subsequent, though not identical, situations. Machine learning (ML) algorithms and statistical models can be used by computer systems to effectively perform a specific task without using explicit instructions, relying on patterns and inference instead. Machine learning algorithms build a mathematical model based on sample data, known as “training data,” in order to make predictions or decisions without being explicitly programmed to perform the task. Machine learning algorithms can be used when it is infeasible to develop an algorithm of specific instructions for performing the task.

For example, supervised learning algorithms build a mathematical model of a set of data that contains both the inputs and the desired outputs. The data is known as training data and consists of a set of training examples. Each training example has one or more inputs and a desired output, also known as a supervisory signal. In the case of semi-supervised learning algorithms, some of the training examples are missing the desired output. In the mathematical model, each training example is represented by an array or vector, and the training data by a matrix. Through iterative optimization of an objective function, supervised learning algorithms learn a function that can be used to predict the output associated with new inputs. An optimal function will allow the algorithm to correctly determine the output for inputs that were not a part of the training data. An algorithm that improves the accuracy of its outputs or predictions over time is said to have learned to perform that task. Supervised learning algorithms include classification and regression. Classification algorithms are used when the outputs are restricted to a limited set of values, and regression algorithms are used when the outputs may have any numerical value within a range. Similarity learning is an area of supervised machine learning closely related to regression and classification, but the goal is to learn from examples using a similarity function that measures how similar or related two objects are.

Reinforcement learning is an area of machine learning concerned with how software agents ought to take actions in an environment so as to maximize some notion of cumulative reward. Due to its generality, the field is studied in many other disciplines, such as game theory, control theory, operations research, information theory, simulation-based optimization, multi-agent systems, swarm intelligence, statistics and genetic algorithms. In machine learning, the environment is typically represented as a Markov Decision Process (MDP). Many reinforcement learning algorithms use dynamic programming techniques. Reinforcement learning algorithms do not assume knowledge of an exact mathematical model of the MDP and are used when exact models are infeasible.

In predictive modeling and other types of data analytics, a single model based on one data sample can have biases, high variability or outright inaccuracies that can affect the reliability of its analytical findings. By combining different models or analyzing multiple samples, the effects of those limitations can be reduced to provide better information. As such, ensemble methods can use multiple machine learning algorithms to obtain better predictive performance than could be obtained from any of the constituent learning algorithms alone.

An ensemble is a supervised learning algorithm because it can be trained and then used to make predictions. The trained ensemble, therefore, represents a single hypothesis that is not necessarily contained within the hypothesis space of the models from which it is built. Thus, ensembles can be shown to have more flexibility in the functions they can represent. An ensemble model can include a set of individually trained classifiers (such as neural networks or decision trees) whose predictions are combined.

For instance, one common example of ensemble modeling is a random forest model which is a type of analytical model that leverages multiple decision trees and is designed to predict outcomes based on different variables and rules. A random forest model blends decision trees that may analyze different sample data, evaluate different factors or weight common variables differently. The results of the various decision trees are then either converted into a simple average or aggregated through further weighting. The emergence of Hadoop and other big data technologies has allowed greater volumes of data to be stored and analyzed, which can allow analytical models to be run on different data samples.

Depending on the implementation, any number of machine learning models can be combined to optimize the ensemble model. Examples of machine learning algorithms or models that can be implemented at the machine learning model can include, but are not limited to: regression models such as linear regression, logistic regression, and K-means clustering; one or more decision tree models (e.g., a random forest model); one or more support vector machines; one or more artificial neural networks; one or more deep learning networks (e.g., at least one recurrent neural network, sequence to sequence mapping using deep learning, sequence encoding using deep learning, etc.); fuzzy logic based models; genetic programming models; Bayesian networks or other Bayesian techniques, probabilistic machine learning models; Gaussian processing models; Hidden Markov models; time series methods such as Autoregressive Moving Average (ARMA) models, Autoregressive Integrated Moving Average (ARIMA) models, Autoregressive conditional heteroskedasticity (ARCH) models; generalized autoregressive conditional heteroskedasticity (GARCH) models; moving-average (MA) models or other models; and heuristically derived combinations of any of the above, etc. The types of machine learning algorithms differ in their approach, the type of data they input and output, and the type of task or problem that they are intended to solve.

A Hidden Markov model (HMM) is a statistical Markov model in which the system being modeled is assumed to be a Markov process with unobserved (hidden) states. An MINI can be considered as the simplest dynamic Bayesian network. A Bayesian network, belief network or directed acyclic graphical model is a probabilistic graphical model that represents a set of random variables and their conditional independence with a directed acyclic graph (DAG). Bayesian networks that model sequences of variables are called dynamic Bayesian networks. Generalizations of Bayesian networks that can represent and solve decision problems under uncertainty are called influence diagrams.

Support vector machines (SVMs), also known as support vector networks, are a set of related supervised learning methods used for classification and regression. Given a set of training examples, each marked as belonging to one of two categories, an SVM training algorithm builds a model that predicts whether a new example falls into one category or the other. An SVM training algorithm is a non-probabilistic, binary, linear classifier. In addition to performing linear classification, SVMs can efficiently perform a non-linear classification using what is called the kernel trick, implicitly mapping their inputs into high-dimensional feature spaces.

Decision tree learning uses a decision tree as a predictive model to go from observations about an item (represented in the branches) to conclusions about the item's target value (represented in the leaves). Tree models where the target variable can take a discrete set of values are called classification trees; in these tree structures, leaves represent class labels and branches represent conjunctions of features that lead to those class labels. Decision trees where the target variable can take continuous values (typically real numbers) are called regression trees. In decision analysis, a decision tree can be used to visually and explicitly represent decisions and decision making.

Deep learning algorithms can refer to a collection of algorithms used in machine learning, that are used to model high-level abstractions and data through the use of model architectures, which are composed of multiple nonlinear transformations. Deep learning is a specific approach used for building and training neural networks. Deep learning consists of multiple hidden layers in an artificial neural network. Examples of deep learning algorithms can include, for example, Siamese networks, transfer learning, recurrent neural networks (RNNs), long short term memory (LSTM) networks, convolutional neural networks (CNNs), transformers, etc. For instance, deep learning approaches can make use of autoregressive Recurrent Neural Networks (RNN), such as the long short-term memory (LSTM) and the Gated Recurrent Unit (GRU). One neural network architecture for time series forecasting using RNNs (and variants) is an autoregressive seq2seq neural network architecture, which acts as an autoencoder.

In some embodiments, the ensemble model can include one or more deep learning algorithms. It should be noted that any number of different machine learning techniques may also be utilized. Depending on the implementation, the ensemble model can be implemented as a bootstrap aggregating ensemble algorithm (also referred to as a bagging classifier method), as a boosting ensemble algorithm or classifier algorithm, as a stacking ensemble algorithm or classifier algorithm, as bucket of models ensemble algorithms, as Bayes optimal classifier algorithms, as Bayesian parameter averaging algorithms, as Bayesian model combination algorithms, etc.

Bootstrap aggregating, often abbreviated as bagging, involves having each model in the ensemble vote with equal weight. In order to promote model variance, bagging trains each model in the ensemble using a randomly drawn subset of the training set. As an example, the random forest algorithm combines random decision trees with bagging to achieve very high classification accuracy. A bagging classifier or ensemble method creates individuals for its ensemble by training each classifier on a random redistribution of the training set. Each classifier's training set can be generated by randomly drawing, with replacement, N examples—where N is the size of the original training set; many of the original examples may be repeated in the resulting training set while others may be left out. Each individual classifier in the ensemble is generated with a different random sampling of the training set. Bagging is effective on “unstable” learning algorithms (e.g., neural networks and decision trees), where small changes in the training set result in large changes in predictions.

By contrast, boosting involves incrementally building an ensemble by training each new model instance to emphasize the training instances that previous models mis-classified. In some cases, boosting has been shown to yield better accuracy than bagging, but it also tends to be more likely to over-fit the training data. A boosting classifier can refer to a family of methods that can be used to produce a series of classifiers. The training set used for each member of the series is chosen based on the performance of the earlier classifier(s) in the series. In boosting, examples that are incorrectly predicted by previous classifiers in the series are chosen more often than examples that were correctly predicted. Thus, boosting attempts to produce new classifiers that are better able to predict examples for which the current ensemble's performance is poor. A common implementation of boosting is Adaboost, although some newer algorithms are reported to achieve better results.

Stacking (sometimes called stacked generalization) involves training a learning algorithm to combine the predictions of several other learning algorithms. Stacking works in two phases: multiple base classifiers are used to predict the class, and then a new learner is used to combine their predictions with the aim of reducing the generalization error. First, all of the other algorithms are trained using the available data, then a combiner algorithm is trained to make a final prediction using all the predictions of the other algorithms as additional inputs. If an arbitrary combiner algorithm is used, then stacking can theoretically represent any of the ensemble techniques described in this article, although, in practice, a logistic regression model is often used as the combiner.

A “bucket of models” is an ensemble technique in which a model selection algorithm is used to choose the best model for each problem. When tested with only one problem, a bucket of models can produce no better results than the best model in the set, but when evaluated across many problems, it will typically produce much better results, on average, than any model in the set. One common approach used for model-selection is cross-validation selection (sometimes called a “bake-off contest”). Cross-validation selection can be summed up as try them all with the training set and pick the one that works best. Gating is a generalization of Cross-Validation Selection. It involves training another learning model to decide which of the models in the bucket is best-suited to solve the problem. Often, a perceptron is used for the gating model. It can be used to pick the “best” model, or it can be used to give a linear weight to the predictions from each model in the bucket. When a bucket of models is used with a large set of problems, it may be desirable to avoid training some of the models that take a long time to train. Landmark learning is a meta-learning approach that seeks to solve this problem. It involves training only the fast (but imprecise) algorithms in the bucket, and then using the performance of these algorithms to help determine which slow (but accurate) algorithm is most likely to do best.

The Bayes optimal classifier is a classification technique. It is an ensemble of all the hypotheses in the hypothesis space. On average, no other ensemble can outperform it. The naive Bayes optimal classifier is a version of this that assumes that the data is conditionally independent on the class and makes the computation more feasible. Each hypothesis is given a vote proportional to the likelihood that the training dataset would be sampled from a system if that hypothesis were true. To facilitate training data of finite size, the vote of each hypothesis is also multiplied by the prior probability of that hypothesis. The hypothesis represented by the Bayes optimal classifier, however, is the optimal hypothesis in ensemble space (the space of all possible ensembles.

Bayesian parameter averaging (BPA) is an ensemble technique that seeks to approximate the Bayes optimal classifier by sampling hypotheses from the hypothesis space and combining them using Bayes' law. Unlike the Bayes optimal classifier, Bayesian model averaging (BMA) can be practically implemented. Hypotheses are typically sampled using a Monte Carlo sampling technique such as MCMC. For example, Gibbs sampling may be used to draw hypotheses that are representative of a distribution. It has been shown that under certain circumstances, when hypotheses are drawn in this manner and averaged according to Bayes' law, this technique has an expected error that is bounded to be at most twice the expected error of the Bayes optimal classifier.

Bayesian model combination (BMC) is an algorithmic correction to Bayesian model averaging (BMA). Instead of sampling each model in the ensemble individually, it samples from the space of possible ensembles (with model weightings drawn randomly from a Dirichlet distribution having uniform parameters). This modification overcomes the tendency of BMA to converge toward giving all of the weight to a single model. Although BMC is somewhat more computationally expensive than BMA, it tends to yield dramatically better results. The results from BMC have been shown to be better on average (with statistical significance) than BMA, and bagging. The use of Bayes' law to compute model weights necessitates computing the probability of the data given each model. Typically, none of the models in the ensemble are exactly the distribution from which the training data were generated, so all of them correctly receive a value close to zero for this term. This would work well if the ensemble were big enough to sample the entire model-space, but such is rarely possible. Consequently, each pattern in the training data will cause the ensemble weight to shift toward the model in the ensemble that is closest to the distribution of the training data. It essentially reduces to an unnecessarily complex method for doing model selection. The possible weightings for an ensemble can be visualized as lying on a simplex. At each vertex of the simplex, all of the weight is given to a single model in the ensemble. BMA converges toward the vertex that is closest to the distribution of the training data. By contrast, BMC converges toward the point where this distribution projects onto the simplex. In other words, instead of selecting the one model that is closest to the generating distribution, it seeks the combination of models that is closest to the generating distribution. The results from BMA can often be approximated by using cross-validation to select the best model from a bucket of models. Likewise, the results from BMC may be approximated by using cross-validation to select the best ensemble combination from a random sampling of possible weightings.

Referring again to FIG. 1, based on the analysis of the standardized structured data, the platform 150 can further generate personalized dietary and health advice or recommendations for each of a plurality of individual users.

Data can enter the platform 150 by connecting to the platform using an API gateway. Accordingly, data can be collected and aggregated from the plurality of different sources through a plurality of Application Programming Interfaces (APIs) connecting to the platform via the API gateway. Data can be aggregated, processed and stored in the platform using autonomous functions, which are triggered as incoming data is streamed through different modules or components within the platform. The components/modules within the platform can be decoupled from one another, thereby ensuring ease of maintenance, updates, and reusability. The processing of the data by the platform is not subject to changes or updates to the underlying APIs. The use of a serverless architecture in the platform can permit data to be processed without loss of data as changes or updates are made to the underlying APIs.

The platform 150 can be configured to handle large volumes of streaming data in or near real time. In some embodiments, the platform can collect, aggregate and process data from a plurality of different sources. The data may comprise at least on the order of 106 daily data points that are evenly or unevenly distributed throughout a day. Some of the data may be retrieved from the API gateway on the order of milliseconds. In some instances, the platform 150 may receive bulk data that is not capable of being processed in real time. The platform may output data in a file format such as Parquet, which is configured to allow for analysis of large data volumes.

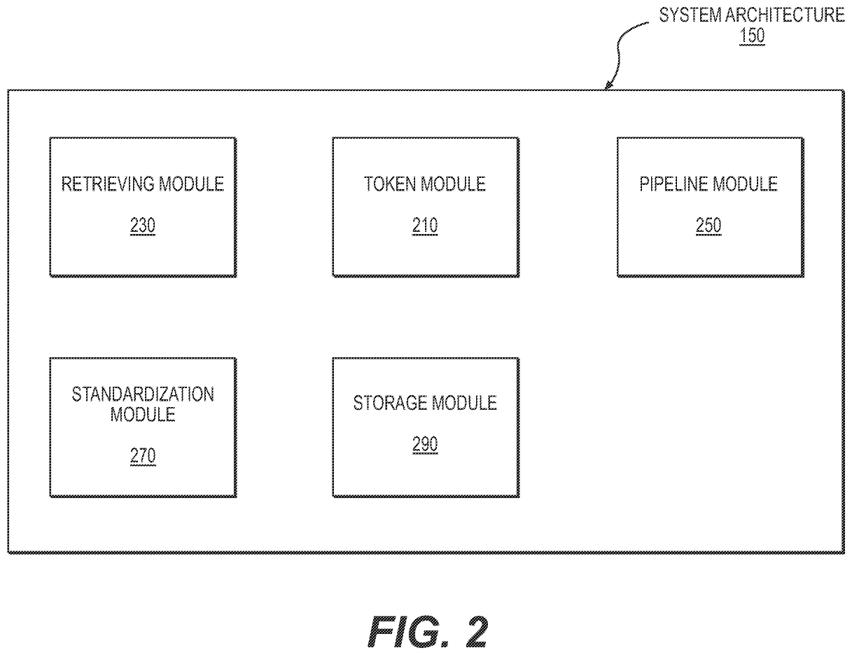

FIG. 2 shows a block diagram of the platform 150 in accordance with some embodiments. The platform may include a token module 210, a retrieving module 230, a pipeline module 250, a standardization module 270, and a storage module 290. The modules may represent groupings of functions, storage units, or applications that can authenticate, direct, store, or process data. Data can flow through the modules serially, but autonomous functions provided within each component may be arranged in a serial or parallel fashion. APIs connected to the platform can be authenticated and be authorized using the token module 210. The retrieving module 230 can be configured to retrieve data from the connected applications. The pipeline module 250 can be configured to direct the data to hosted and third party applications for further processing or storage. The standardization module 270 can process the data and convert the data into a standardized structured format. The standardized structured data can be further analyzed within the platform, for example using one or more machine learning models described herein. Alternatively, the standardized structured data can be exported to one or more third-party applications for analysis. Finally, the storage module 290 can store the processed data, monitor passive data collection, and prepare the data to be used for different types of analysis.

The token module 210 can integrate external APIs and data services, and is responsible for authorizing and authenticating these external APIs. The token module 210 may or may not represent itself as a token module when authenticating third-party applications. For example, the token module 210 may, when communicating with external APIs and data services, represent itself as the platform 150, or as a different service collecting data from third-party applications. This can allow the token module 210 to anonymize the services by third party applications while keeping their identities intact. Accordingly, the token module may be used to manage access to one or more third party applications provided by different entities (e.g., companies).

The token module 210 can create, renew, and delete tokens. The token module can refresh existing tokens and provide notification updates about token changes. Tokens created by the token module 210 can be duplicated and transferred to the retrieving module, which is decoupled from and independent of the token module. Each time a new token is generated, the new token may be separately duplicated in the retrieving module, in addition to being stored in the token module. Created tokens may have expiration dates. In order to maintain permissions, the token module 210 may issue tokens to the retrieving module 230 on a scheduled basis. In some embodiments, the retrieving module 230 may send a message, using a simple notification service, to the token module 210 when it does not have the necessary tokens or if the tokens are not working properly.

The token module 210 may use OAuth to integrate external APIs. A user may log into an application using the platform 150. Using the platform's API, the application may request to initiate an authentication process between the user and one or more additional third party applications. When the user is authenticated by the one or more third party applications, the application using the platform receives an access token, which is stored on the platform 150. The authentication process may use OAuth 1.0 or OAuth 2.0.

The retrieving module 230 can retrieve data from APIs connected to the platform 150. The retrieving module 230 may serve as a hub for the platform to integrate with many types of applications, wearable devices, mobile devices, medical devices, datasets, data sources, among others. The retrieving module can interface with various devices and receive data. The data can be received in a form commonly exported by a corresponding application. The retrieving module 230 may comprise a set of processing functions that can consolidate data from many different applications into a stream. The data can be consolidated in an asynchronous manner, and persist in the stream for a fixed period of time. After the data is received and consolidated into a stream, it can be directed as a data package to other modules for processing. The retrieving module may be decoupled from and independent of other modules. For example, the retrieving module may be only configured to collect and aggregate the data, and may not be configured to persist, store, or process the data.

The retrieving module 230 can be configured to pull data and receive pushed data. Data may be collected directly from connected APIs or received from mobile devices. In these embodiments, data from mobile device applications may be stored in a bucket object, such as an AMAZON® S3 bucket. The pushed and pulled data may be received simultaneously or at different times. The received pushed and pulled data may be moved into data streams. Data may be sent to different modules within the platform using processing functions at various stages. The data stream may comprise data shards, which are queues of data records. Changing the number of shards can change the speed at which the data is processed. Accordingly, the platform can control a speed at which the data is being processed, by controlling a number of shards in the streams. Each data stream may have a retention policy, which dictates how long the data persists in the stream. In some embodiments, data may be retained for a period ranging from 24 hours to 168 hours. Each shard may comprise a string of data records that (1) enter into a queue and (2) exit the queue upon expiration of the retention policy. At any point within the retention period, data records from earlier in the queue can be viewed, and the stream can be reiterated to a previous historical state. After this period has expired, the data records may exit the queue. An individual service may push data from one type and have data of another type pulled by the retrieving module 230. In some embodiments, the string of data records may include food consumption, health or nutrition records that are specific to a plurality of individual users. Pulling data from applications may be performed by a time-based task scheduler on a periodic basis. Examples of applications from which data can be pulled into the platform 150 may include third party applications such as Withings.

The retrieving module 230 may also retrieve data from applications that push data into the platform. Examples of services and devices that can push data into the platform may include Abbott FreeStyle Libre, Garmin, FitBit, and the like. These devices can send notifications to the platform 150, which can be received by the retrieving module 230. In response, the retrieving module 230 can pull data from these applications and consolidate/store the data into the stream or a plurality of streams. In some instances, the data may be sent to the platform concurrently with the notifications, thus eliminating the need for the retrieving module 230 to pull data in response. In some embodiments, the retrieving module 230 can also store tokens created by the token module 210. Tokens may be stored in a serverless database provided with the retrieving module.

The pipeline module 250 can control the flow of data through the platform 150. The pipeline module 250 can facilitate data transfer to third party applications, examples which may include Welltok, Medtronic, and the like. The pipeline module 250 may also transfer data to streaming applications within the platform. Data can be transferred using autonomous functions that are triggered in response to events. Events may include creating an object and receiving a notification message, such as an SNS message. In some embodiments, the pipeline module 250 can be implemented by AMAZON® Kinesis stream using AMAZON® Lambda functions and AMAZON® S3 buckets. Kinesis can trigger Lambda functions that direct data to various resources within the platform, in response to events. An event may be a receipt of a data shard within a stream.

The standardization module 270 can manage and process data streaming through the platform 150, by creating a standardized, consolidated structured data file that can be read by third party applications or have analysis performed on the standardized data file. The standardization module 270 may include a set of elements that can implement different processing functions on the data stream. The processing functions may include sorting, converting the data into different formats, and removing redundant data. Processed data streams may be stored, cached, or directed into additional data streams.

The storage module 290 can manage the platform's passive data collection activities. The management of the data collection activities may include keeping logs of streamed data pulled by a passive data collection SDK, as well as analyzing, classifying, and storing the streamed data. The storage module 290 may automatically perform analysis on data pulled from connected applications or native applications on mobile devices. A mobile device camera may passively collect image data, which may be pushed by a passive data collection software development kit (SDK) to the storage module 290. The storage module 290 may classify the images as either containing food items or not containing food items, using a binary classifier built with a neural network. This data may be pre-processed or encrypted before analysis.

In subsequent figure descriptions, each module within the platform 150 may be described as including one or more components. These components may each include groupings of one or more autonomous functions, data streams, streaming applications, storage buckets, or other components, connected logically and configured as a group to perform a task. Data streamed through the platform 150 may trigger one or more autonomous functions within these groupings. For example, AMAZON® Lambda functions may be triggered using AMAZON® Kinesis data streams or AMAZON® A3 events. Kinesis, for example, can trigger Lambda functions when it detects new records in a stream. Functions may also be triggered in response to a record written into a DynamoDB table. AMAZON® Lambda may poll these sources in order to determine when new records are available.

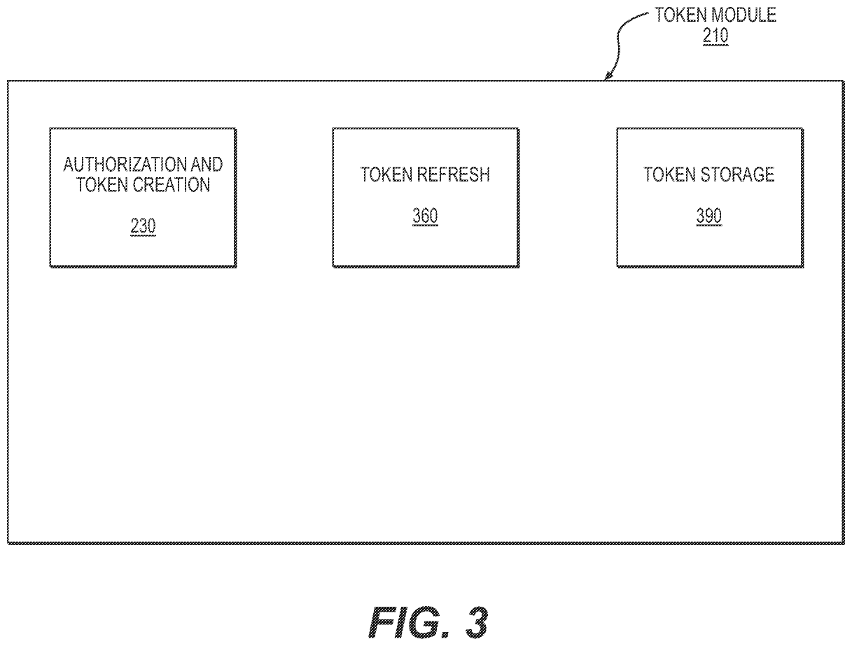

FIG. 3 shows the components of the token module 210 in accordance with some embodiments. The token module 210 may include an authentication and token creation component 330, a token refresh component 360, and a token storage component 390.

The authentication and token creation component 330 may communicate with external APIs. The authentication and token creation component may receive connection requests at a URL and store the requests. The tokens may be user access tokens. Tokens may contain expiration dates, permissions, and identifiers. In some embodiments, the requests may be stored in a table. The authentication and token creation component may also maintain an authorization URL. After the user is authorized, the callback function may return the user to the authorization URL. When an external API is authenticated and authorized, the authorization and token creation component may issue a token, which is stored in the token module 210. Tokens may also be stored in a table. The authorization and token creation component may also duplicate tokens and transfer the duplicate tokens to the receiving module 230. Token information, such as a token's expiration date, may be copied with the token into the receiving module 230. Tokens may also be deleted using component 330 when they expire.

The token refresh component 360 may be a scheduling component that runs periodically, checks expiration dates of existing tokens, and refreshes tokens that are soon to expire. The token refresh component 360 can update subscribers to tokens. Notifications can provide updates about changes in token status. The token refresh component may update components, for example, using a simple notification service. The token storage component 390 can keep a record of the tokens in the platform. When tokens are created or refreshed, they can be stored in the token storage component 390.

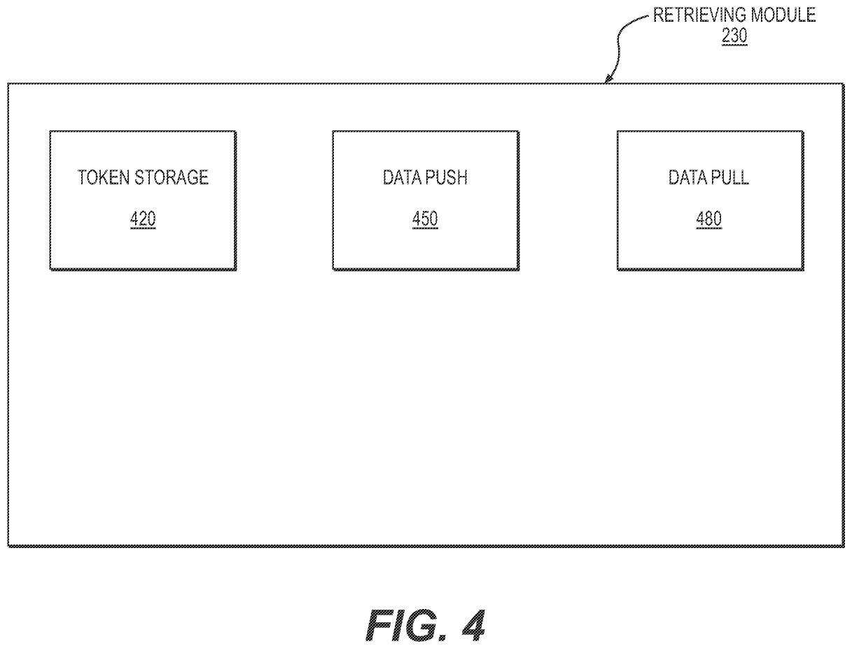

FIG. 4 shows the components of the receiving module 230 in accordance with some embodiments. The receiving module 230 may include a token storage component 420, a data push component 450, a data pull component 480, and a data consolidation component 490.

The token storage component 420 can store tokens created by the token module 210. The token storage component can also retrieve refreshed tokens from the token module 210. When the token module 210 authorizes an API, it can send a message to the retriever module to create a new token. The message may contain the token information. The created token may be stored in a table. Reissued tokens may also be stored in the table. The token storage component 420 may receive notifications from the token module 210 to update tokens that are already stored. For example, the token storage component 420 may receive a command from the token module 420 to update a token's timezone field. The token storage component 420 may receive a notification to replace an existing soon-to-be-expired token with a new token.

The data push component 450 may allow external APIs to push data into the receiving module 230. External APIs may communicate with the receiving module 230 with an API gateway. The data push component 450 may subscribe to notifications from the external APIs. When data is available, the data push component 450 may be prompted to retrieve the data being pushed by an external API. This data may be stored locally. The receiving module 230 may store raw response data in one or more objects, such as one or more storage buckets.

The data pull component 480 may pull data from external APIs. Data may be pulled on a scheduled basis, from APIs that have valid tokens stored on the retrieving module 480. The data pull component 480 may not provide all of the available data for streaming. Instead, the data pull component 480 may submit a partial subset of this data to the data consolidation component 490.

The data consolidation component 490 may place (a) the pushed data from the data push component 450 and (b) the data pulled from the data pull component 480, into data streams. The term “consolidation”, as used herein, may include moving data received from third-party applications into one or more data streams. The data consolidation component may be prompted to move the data into streams via push notification from either the data push component 450, the data pull component 480, or both. The data consolidation component 490 may contain an autonomous function to retrieve historical data from an API (e.g., data collected from a previous month). The historical data function may only be called once, for example, when a new user is enrolled in the platform. Some of the data from one or more data streams may be saved locally. The data consolidation component 490 may provide one or more of the data streams to the other connected modules within the platform.

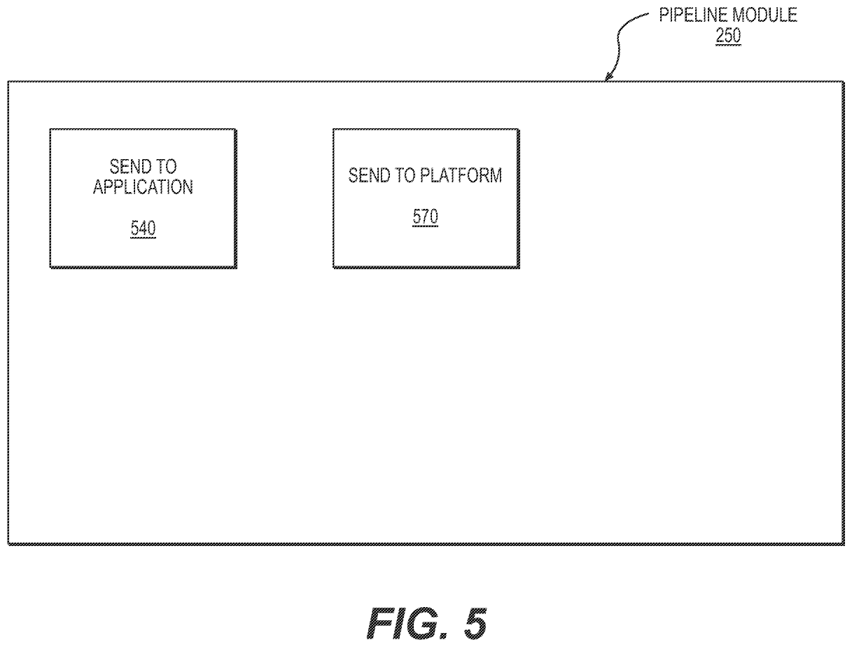

FIG. 5 shows the components of the pipeline module 250 in accordance with some embodiments. The pipeline module 250 may include a component for sending data from the data stream to applications 540 and a component for sending data within the platform 570. The pipeline may leverage a pipeline design pattern, and may include a group of elements connected serially. Example elements may include lambda functions that are used to transfer the streamed data. The lambda functions operating on the streamed data may differ depending on which external API(s) is providing the data, the processing rate, and/or other conditions within the platform. The data being transferred from the stream may be duplicated, in case two or more components of the platform need to process the same data.

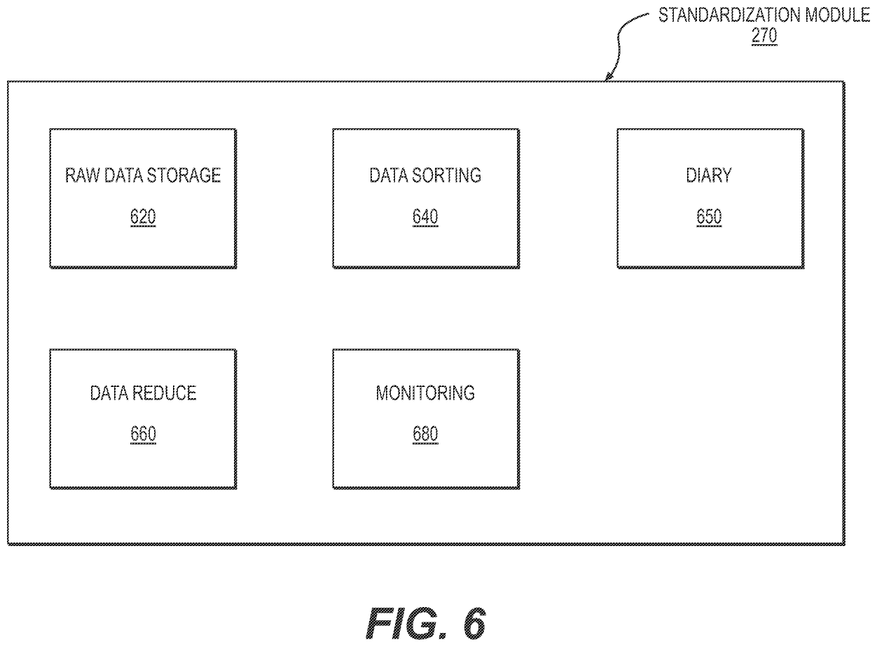

FIG. 6 shows the components of the standardization module 270 in accordance with some embodiments. The standardization module 270 may include a raw data storage module 620, a data sorting module 640, a diary 650, a data reduce module 660, a monitoring module 680, and a converting module 690. In other embodiments, the standardization module 270 may include different or additional data processing components. Components may be arranged serially, so that the stream may be processed in stages by multiple components in succession during preparation for data analysis or storage by the storage module 290. When data is updated and presented to the receiving module 210, the updates may be reflected in the streams in the standardization module 270.

The raw data storage module 620 may store data collected from third party applications. The data being stored may be “raw” data that has yet to undergo processing by the platform components. Such data may be passively collected from applications on mobile devices, such as Apple Health Kit. The raw data may be stored directly into the raw storage module 620, and may bypass the token module 210 and retriever 230.

The data sorting module 640 may sort data received from the pipeline module 250. The data may be sorted by user ID, data type, or activity timestamp. The sorting may be called by a function and performed using an application streaming on the platform. The sorted data may be cached for quick access. The sorted data may be placed in a stream, such as an AMAZON® Kinesis Firehose stream, for easy storage or loading into analysis tools. After sorting, the data may be processed using other tools in the standardization module 270. The data sorting module 640 may also verify, by checking a cache, that data it receives is not duplicate data.

The diary 650 may store standardized, processed data for consumption by third party applications or by end users. The diary may store manually logged data and derived data. Manually logged data may include meals, workouts, self-reported feelings, sleep duration and self-reported quality, height, weight, medications, and insulin levels. Derived data may be calculated from the logged data, and may include metrics such as body fat percentage and basal metabolic rate (BMR). Passively collected data from connected applications may be consolidated with this data and may include synchronized health information from applications and wearable devices such as the Fitbit, Apple Watch, Oura ring and Runkeeper. Individual diary entries may include this consolidated, processed data, converted into a standardized structured format. Entries may be added one at a time or in bulk. Diary entries may be cached for quick access. Diary entries can be used by the platform to create a report providing summary information and recommendations to the user. For example, the diary entries may produce a report containing summary glucose information, meal statistics and tips to improve eating habits, and correlations between blood glucose and physical activity, sleep, and mood.

The data reduce module 660 may remove redundant or extraneous entries from the data stream. The data reduce module 660 may use a Map-Reduce algorithm in order to remove redundant or extraneous entries. For example, the third party application MyFitnessPal may pair nutrients with foods. For each nutrient, MyFitnessPal may list each nutrient within a food. However, this can lead to many duplicate entries, as many nutrients may be contained within the same food item. The data reduce module 660 can consolidate these entries by creating a “food” key and listing each of the food's nutrients as values, so that each food is listed once with its nutritional information.

The monitoring module 680 can ensure that the processing stages of the standardization module 270 are working correctly. In order to do this, the monitoring module 680 may generate dummy data. The dummy data may be placed in a stream, and directed to one or more of the processing modules within the data standardization module. The dummy data produced by the monitoring module 680 may be from one or more data types used in processing. The monitoring module may create a report from testing the dummy data, and provide the report to an external analytics service (such as DATADOG®).

The conversion module 690 may convert the streaming data into other data formats. The file formats into which data is converted may depend on the subsequent processing stages within the standardization module 270. For example, the conversion module 690 may convert the data into a FoodPrint™ data format. Converted streaming data may be stored in a cache for quick access, or transmitted to other processing stages within the standardization module 270.

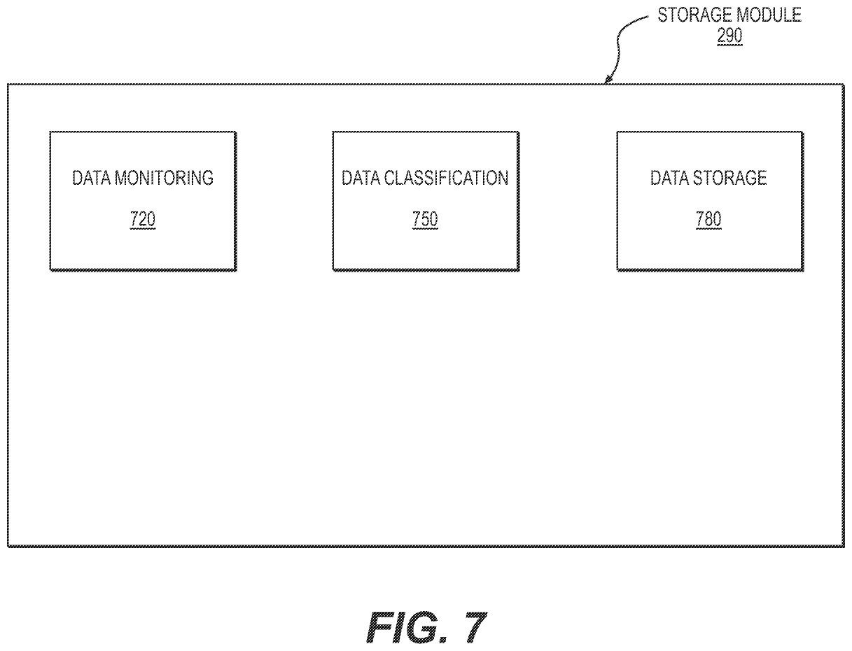

FIG. 7 shows the components of the storage module 290 in accordance with some embodiments. The storage module 290 may include a data monitoring module 720, a data classification module 750, and a data storage module 780. The storage module's components may operate on passively collected data. The passive data may be collected from applications on a user mobile device.

The data monitoring module 720 may include one or more lambda functions that receive data reported from different components within the platform in order to monitor the components. The data may be collected by the components from external devices. One of the lambda functions may invoke an application that can print information about data being collected. The monitoring process may analyze different actions performed on passively-collected data, including obtaining the data, saving the data, packaging the data, encrypting the data, uploading the data, and saving data to one or more servers. An additional lambda function may save information collected through the monitoring process in a log file, which may be stored at a URL.

The data classification module 750 may receive encrypted files that contain passively collected data. The data classification module 750 may contain one or more lambda functions that perform pre-processing on these files before the collected data can be classified. Pre-processing activities may include unzipping and decrypting the files. The data classification module may use lambda functions to classify the collected data. Classification may involve using machine learning or deep learning techniques, such as convolutional or recurrent neural networks. For example, image recognition analysis may be performed on images taken with a mobile phone camera to determine whether or not food exists anywhere in those images. Images containing food may be stored in the diary 650, where nutrition information may be extracted from these images. After classification has been completed, classified data may be stored in a debug bucket in order to troubleshoot the classifier. The data classification module 750 may implement one or more security policies to ensure that data is anonymized in case the data is compromised or stolen. For example, faces of people in images may be blurred. Data may also be encrypted if it is to be uploaded to the cloud.

The data storage module 780 may store passive data that has been classified. Stored data can be analyzed by third party applications, and can provide users with analytics (such as geolocation, file resolution, and camera module data) to improve data analysis models. Passive data that is stored may include image data as well as logged information. Logged information may include manually input or autologged sleep and activity information from third party applications. Classified data may be stored in the data storage module 290 temporarily, and may be deleted after a fixed period of time.

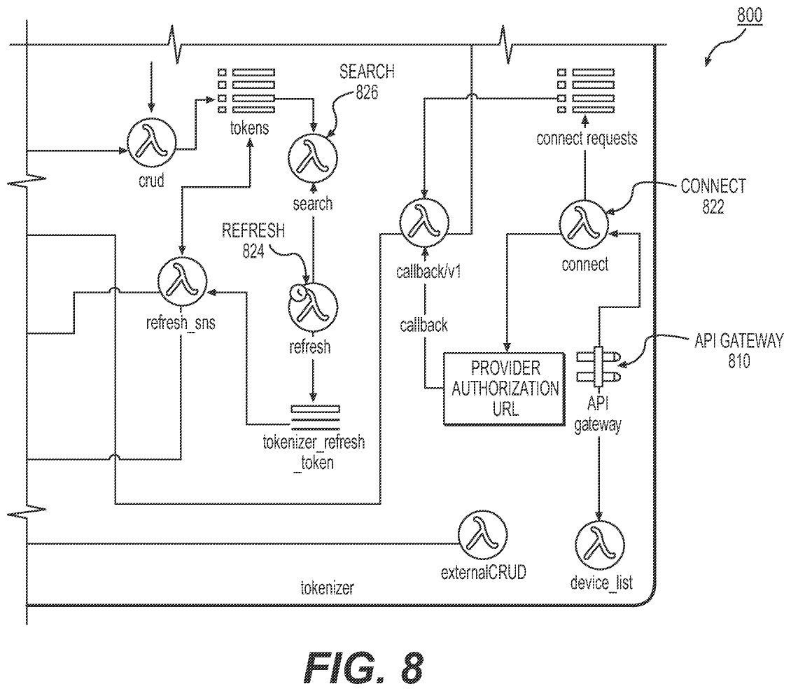

FIGS. 8-12 show example embodiments of the modules within the platform 150.

The example embodiments may employ AMAZON® Web services and AMAZON® Lambda serverless computing. Other serverless architectures, such as GOOGLE® Cloud Functions and MICROSOFT® Azure, may also be used to create the infrastructure disclosed herein. If a platform is developed using a similar type of serverless architecture, the components of the platform may be analogous to the embodiments described herein. The elements in these diagrams may include Lambda functions, API gateways, web servers, simple notification service (SNS) messages, storage buckets, Kinesis Firehose streams, and streaming applications.

Lambda functions may be autonomous functions, as implemented using AMAZON® Lambda. These are functions that are triggered in response to events and are only active when being called. These functions can reduce the amount of time that server resources need to be active. Lambda functions can be used to implement authentication, authorization, data transfer, processing, and storage functions. One or more of the aforementioned functions may be implemented using one or more Lambda functions. For example, a Lambda function may be used to authenticate an API gateway and request a token from an authorization server. Another Lambda function may be used to redirect an authorized API to a URL. Additional Lambda functions may issue, refresh, and delete tokens. Similarly, different Lambda functions may be used to pull or push data, consolidate the pulled and pushed data, and direct different data items from the stream to different places. Lambda functions can integrate with many types of AMAZON® objects, including data streams, data storage, and streaming applications.

API gateways can connect the platform with external APIs in order to transfer data from external applications to the platform. The API gateway also an external entry point for the entire OAuth process to integrate external APIs and data services. The platform can also subscribe to APIs that push data through an API gateway, and thus receive notifications through the gateway when data is ready to be pushed. API gateways can also allow the external APIs to receive data from the platform itself.

Application APIs can allow transfer of and interaction with data by using HTTP calls. APIs may follow REST, which defines a set of rules for interacting with these APIs. They can POST data to resources, GET data from resources, update data in a resource, or delete data from a resource by sending request messages. These messages may include text fields with the message, the user, the credentials, the timestamp, and other message information. The API gateway may communicate with applications using HTTP requests.

Web servers may store resources, such as HTTP resources, and may be connected to the platform via a network. Web servers may store authorization information, and may issue tokens to the platform in order to allow the platform to access user data from external APIs. Web servers may also store processed information. Applications running on the platform 150 may access data stored on web servers. Streaming applications may be hosted on web servers.

AMAZON® S3 buckets may allow users and applications to store data. Data objects can be uploaded and downloaded from buckets. Buckets may also contain metadata, which gives information about the fields stored in them. Buckets can restrict or allow access to users or applications by modifying their permissions. The platform 150 may retrieve data from buckets for processing and consolidation with streams from the retriever 230.

AMAZON® Kinesis streams can load streaming data onto other tools. They can also encrypt and transform the data. Firehose streams can be used in conjunction with Lambda functions to direct data to storage or applications for processing. Kinesis can batch and compress the data that is to be stored, minimize the amount of storage that needs to be used. It can increase security by encrypting the streaming data.

The streaming applications can be hosted by AMAZON® Web Service, and can run securely and with high performance. Applications may be used in an on-demand fashion, as needed, as data is being transferred to them. Streaming applications can be paired with Lambda functions that direct the data after the data has been processed.

FIG. 8 shows an embodiment 800 of the token module 210 in accordance with some embodiments. The token module may include an API gateway 810 that connects the platform 150 to external APIs. A lambda function connect 822 may save connection requests from external APIs in a table. External APIs may be authenticated and authorized by lambda functions. Following authorization, a lambda function may be invoked to create a token. In the embodiment illustrated, a lambda called refresh 824 may send a message to refresh a token. Another lambda 826 may search the database for existing tokens. Tokens may be issued to the retrieving module 230. A lambda disable 828 may also be invoked to disable one or more tokens stored on the retrieving module. Another lambda may be used for create, read, update, and delete (CRUD) operations.

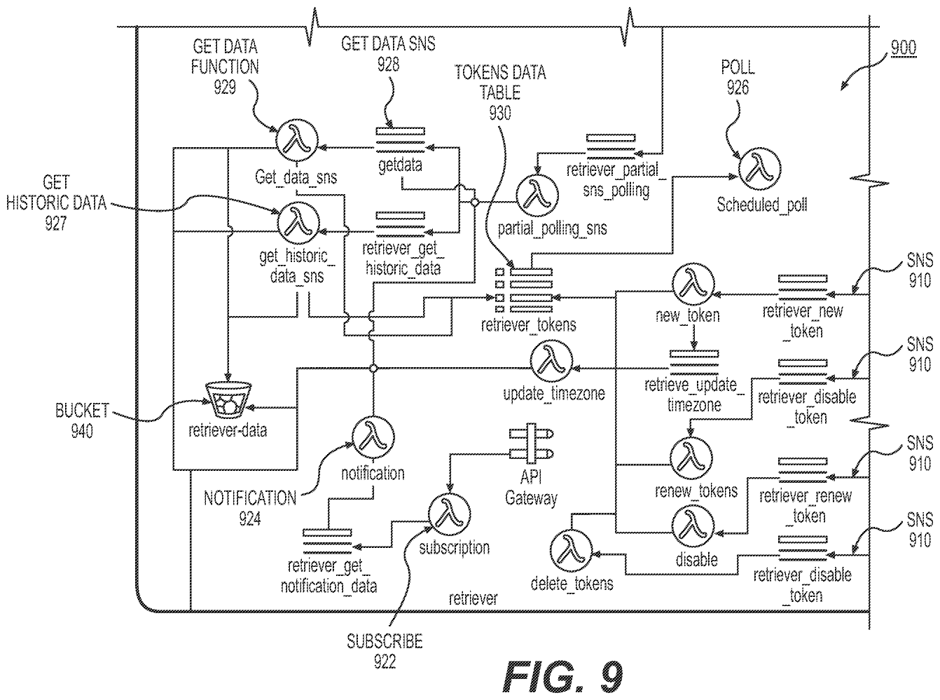

FIG. 9 shows an embodiment 900 of the retrieving module 230. In the example of FIG. 9, the retrieving module may receive messages from the token module embodiment 800 to renew, create, and disable tokens. The messages may be simple notification service (SNS) messages 910. The tokens may be updated in the tokens data table 930. In Referring to the data pushing component 450 in FIG. 9, the retriever may connect to an API gateway in order to receive data pushed from connected external APIs. The subscribe 922 lambda may send an SNS message to a notification 924 function, which indicates the data that is to be pushed into the retrieving module embodiment 900. Referring to the data pulling component 480 in FIG. 9, the function scheduled_poll 926 may poll the connected external APIs, as listed in the tokens data table 930. The SNS message getdata 928 may announce that new data is available, and the pushed and pulled data may be retrieved using the lambda function get_data_sns 929. The function get_historic_data_sns 927 may receive data from an earlier timepoint in the retention period and add to the data stream. The bucket retriever-data 940 may store data sent into the retriever, for debugging or backup.

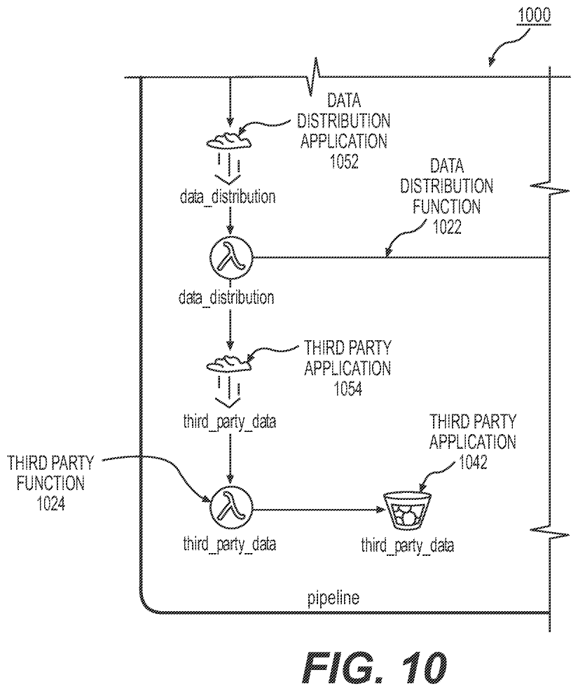

FIG. 10 shows an embodiment 1000 of the pipeline module 250. In this embodiment, two streaming applications and two lambda functions may be connected serially. Data from the retriever module embodiment 900 may be sent to a streaming application called data_distribution 1052. A lambda function data_distribution 1022 may direct this data to the standardization module embodiment 1100. Another streaming application third_party_data 1054 and a corresponding lambda function 1024 may send the stream to the third-party application bucket 1042.

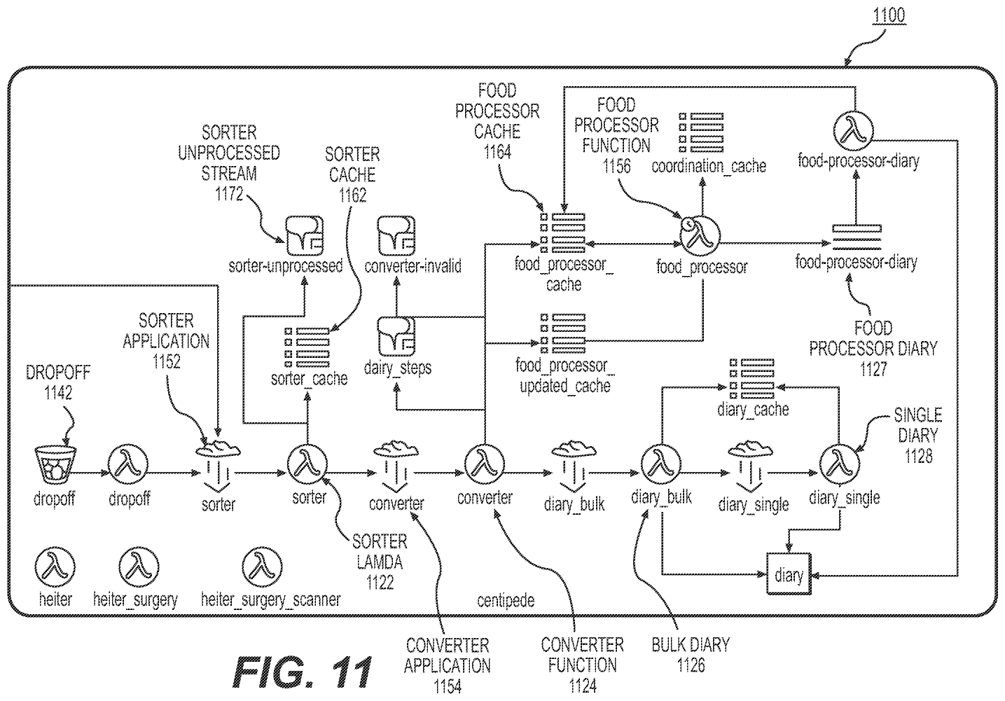

FIG. 11 shows an embodiment 1100 of the standardization module 270. This embodiment may include a serial processing chain, as well as multiple caches and AMAZON® Kinesis streams at various stages of the serial processing chain. The data may be stored in AMAZON® S3 buckets using, for example, AMAZON® Kinesis Firehose. Streaming data may be received from a storage area called Dropoff 1142 as well as from the pipeline embodiment 900. The data may be sorted by a sorter lambda 1152, and directed by a corresponding lambda 1122 to be saved in both a sorter_cache 1162 and a sorter-unprocessed stream 1172. The data may then be converted using a converter function 1154 and directed to a diary resource with the converter lambda function 1124. The converted data may be saved in a diary by the diary_bulk 1126 function and cached. Single diary entries may be extracted by the diary_single 1128 function and stored in the diary. The converted data may also be stored in two food_processor_caches 1164. The data may be reduced by the lambda_function food_processor 1156 and directed by a function food_processor_diary 1127 into the diary. The coordination_cache function acts as a mutex to keep functions from executing in parallel.

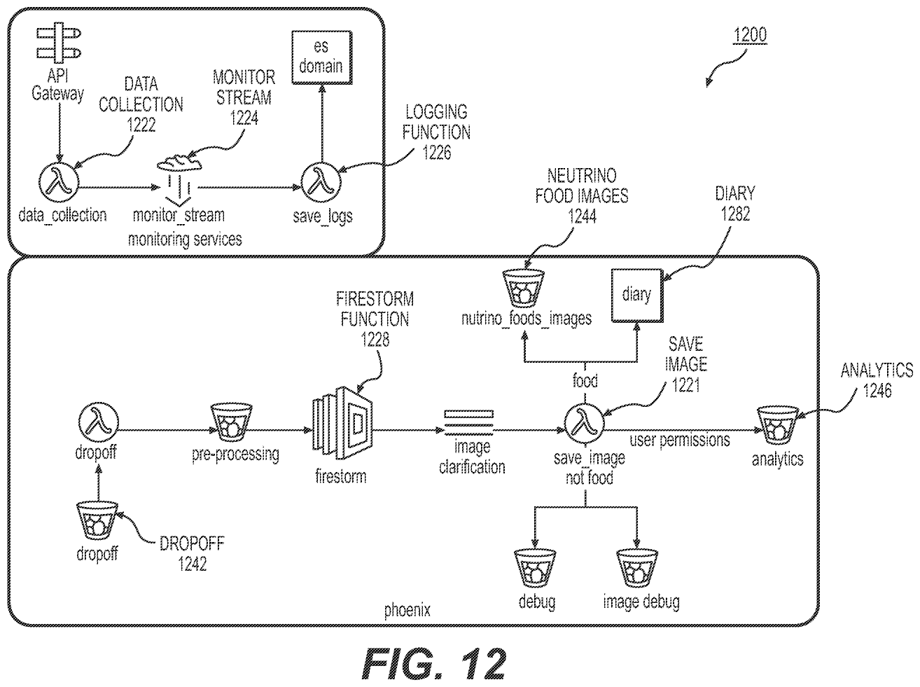

FIG. 12 shows an embodiment 1200 of the storage module 290. The data monitoring module may monitors data from system components using the function data_collection 1222. The stream may be monitored by the application monitor_stream 1224. An additional lambda function 1226 may save logs from the monitoring process to es domain. The data classification module may collect data from “dropoff” 1242, pre-process the data using the lambda function firestorm 1228, and classify food images. The lambda function save_image 1221 may place the items that have not been classified as food items into debug and image debug buckets. The images that are classified as food may be saved by the storage module in food_images 1244, a diary 1282, and an analytics bucket 1246.

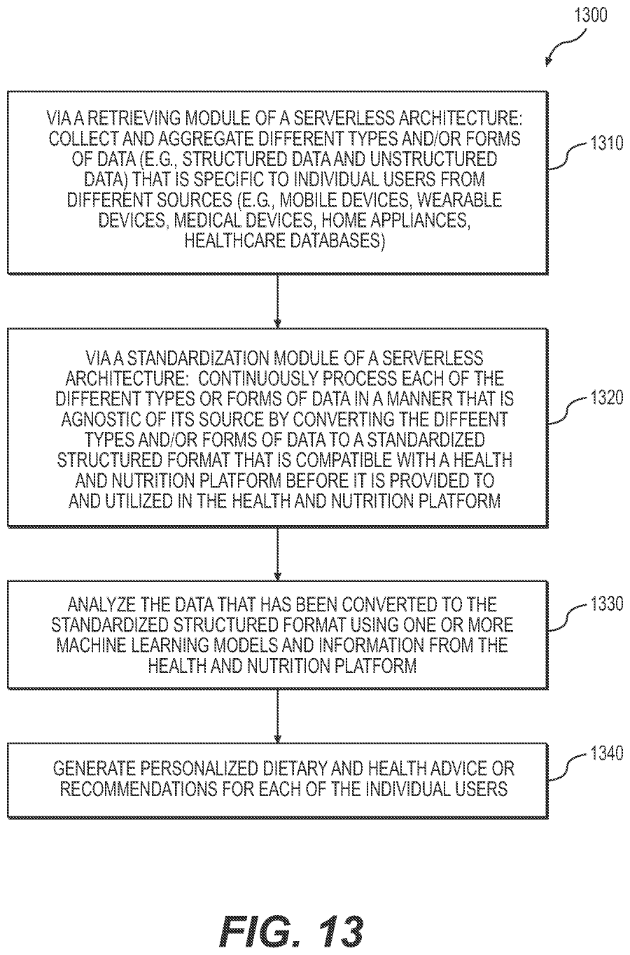

FIGS. 13-18 are flow charts that illustrates examples of methods in accordance with the disclosed embodiments. With respect to FIGS. 13-18, the steps of each method shown are not necessarily limiting. Steps can be added, omitted, and/or performed simultaneously without departing from the scope of the appended claims. Each method may include any number of additional or alternative tasks, and the tasks shown need not be performed in the illustrated order. Each method may be incorporated into a more comprehensive procedure or process having additional functionality not described in detail herein. Moreover, one or more of the tasks shown could potentially be omitted from an embodiment of each method as long as the intended overall functionality remains intact. Further, each method is computer-implemented in that various tasks or steps that are performed in connection with each method may be performed by software, hardware, firmware, or any combination thereof. For illustrative purposes, the following description of each method may refer to elements mentioned above in connection with FIG. 1. In certain embodiments, some or all steps of this process, and/or substantially equivalent steps, are performed by execution of processor-readable instructions stored or included on a processor-readable medium that is or can be non-transitory. For instance, in the description of FIGS. 13-18 that follows, various components of the platform 150 (e.g., a token module 210, a retrieving module 230, a pipeline module 250, a standardization module 270, a storage module 290 and any components thereof) can be described as performing various acts, tasks or steps, but it should be appreciated that this refers to processing system(s) of these entities executing instructions to perform those various acts, tasks or steps. Depending on the implementation, some of the processing system(s) can be centrally located, or distributed among a number of server systems that work together.

FIG. 13 is a flowchart that illustrates a computer-implemented data collection and processing method 1300 that is implemented using a serverless architecture including a health and nutrition platform 150 for generating personalized dietary and health advice or recommendations via a hardware-based processing system in accordance with the disclosed embodiments. The method 1300 begins at 1310 where a retrieving module 230 collects and aggregates data from a plurality of different sources in a storage module 290. The data can include different types or forms of data (e.g., structured data and unstructured data that comprises food, health or nutritional data that is specific to a plurality of individual users).

At 1320, a standardization module 270 can continuously processes each of the different types or forms of data in a manner that is agnostic of its source, for example, by converting the different types or forms of data to a standardized structured format that is compatible with the health and nutrition platform 150.

At 1330, the data that has been converted to the standardized structured format can be analyzed using (at least in part) information from the health and nutrition platform 150. For example, the standardized structured data can be analyzed using one or more machine learning models including, but not limited to, one or more artificial neural networks; one or more regression models; one or more decision tree models; one or more support vector machines; one or more Bayesian networks; one or more probabilistic machine learning models; one or more Gaussian processing models; one or more Hidden Markov models; and one or more deep learning networks. At 1340, the personalized dietary and health advice or recommendations for each of a plurality of individual users can be generated.

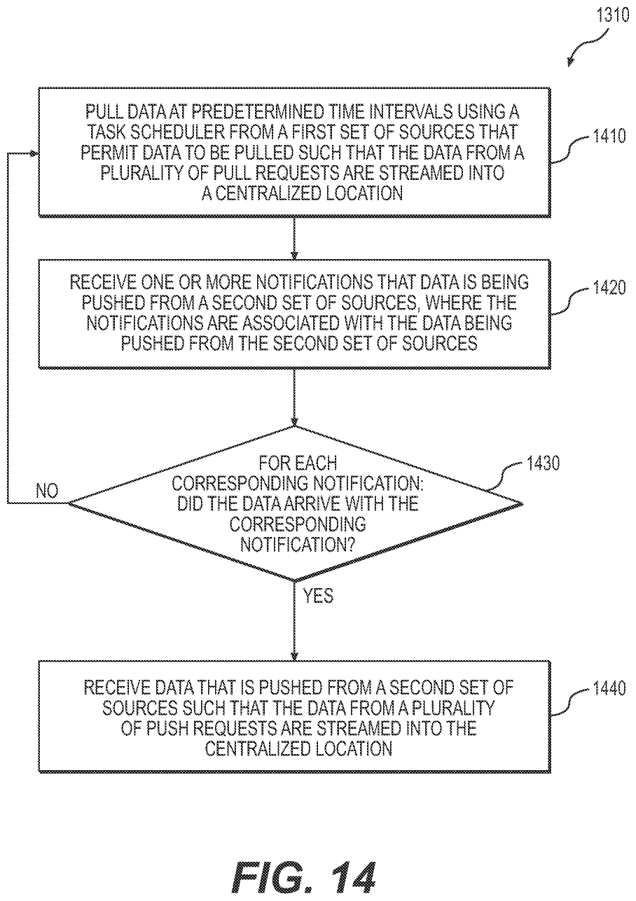

FIG. 14 is a flowchart that illustrates a method 1310 for collecting and aggregating of the data from the plurality of different sources in accordance with the disclosed embodiments. The method 1300 begins at 1410 where data is pulled from a first set of sources that permit data at predetermined time intervals using a task scheduler. At 1420, one or more notifications associated with data that is being pushed from a second set of sources can be received. At 1430, it can be determined whether the data for each corresponding notification has arrived with the corresponding notification. When it is determined (at 1430) that the data for each corresponding notification has not arrived with the corresponding notification, then the method 1310 proceeds to 1410, where data associated with a corresponding notification is pulled if that data does not arrive with the corresponding notification. When it is determined (at 1430) that the data for each corresponding notification has not arrived with the corresponding notification, then the method 1310 proceeds to 1440, where data that is pushed from the second set of sources can be received. Data from a plurality of the push requests can be streamed into a centralized location.

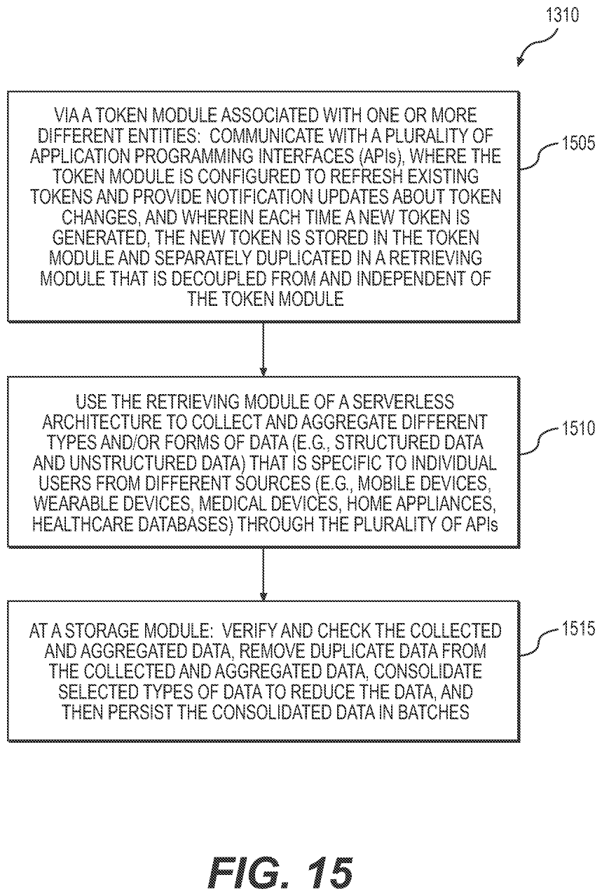

FIG. 15 is a flowchart that illustrates a method 1310 for collecting and aggregating data from a plurality of different sources in a storage module 290 in accordance with the disclosed embodiments. The method 1310 begins at 1505, where a token module 210 associated with one or more different entities can communicate with a plurality of Application Programming Interfaces (APIs) associated with one or more different entities. The token module 210 can refresh existing tokens and provide notification updates about token changes. Each time a new token is generated, the new token is separately duplicated at the retrieving module 230 in addition to being stored in the token module 210. The retrieving module 230 is decoupled from and independent of the token module 210.

At 1510, the retrieving module 230 can collect and aggregate different types or forms of data (e.g., structured data and unstructured data that comprises food, health or nutritional data) that is specific to a plurality of individual users from the plurality of different sources through a plurality of APIs associated with one or more different entities.

At 1515, the storage module 290 can verify and check the collected and aggregated data, remove duplicate data from the collected and aggregated data, consolidate selected types of the collected and aggregated data, reduce the collected and aggregated data and persist the consolidated data in batches

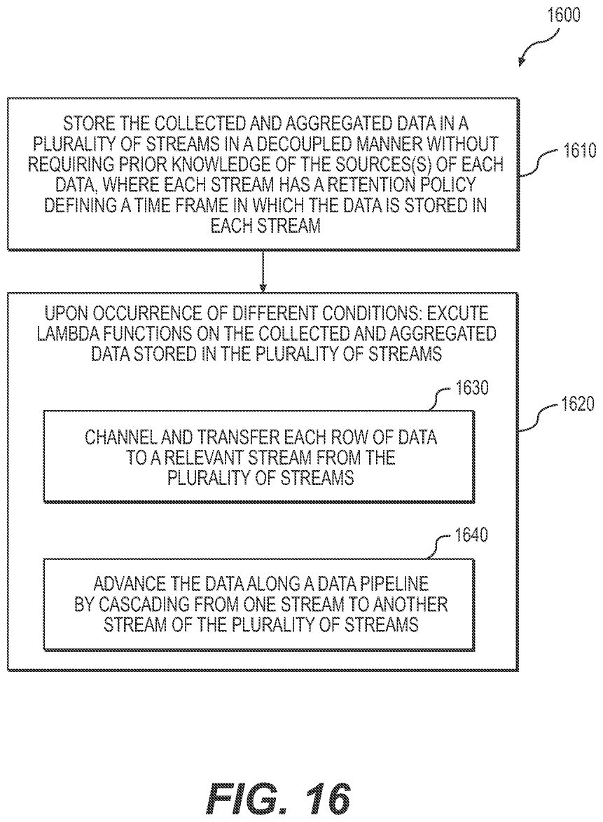

FIG. 16 is a flowchart that illustrates a method 1600 for storing collected and aggregated data from a plurality of different sources and processing the collected and aggregated data in accordance with the disclosed embodiments. The method 1600 begins at 1610 where a retrieving module 230 stores collected and aggregated data from a plurality of different sources in a plurality of streams at a storage module 290. In one embodiment, the plurality of streams each have a retention policy defining a time frame in which the data is stored in each stream. At 1620, upon occurrence of different conditions, the collected and aggregated data can be processed by executing lambda functions on the collected and aggregated data that is stored in the plurality of streams. In one embodiment of 1620, the lambda functions are executed only when the data is collected and stored in the plurality of streams. For example, at 1630, the lambda functions are executed on the stored data at 1630 to channel and transfer each row of data to a relevant stream from the plurality of streams, and at 1640, the collected and aggregated data is advanced along a data pipeline by cascading from one stream to another stream of the plurality of streams.

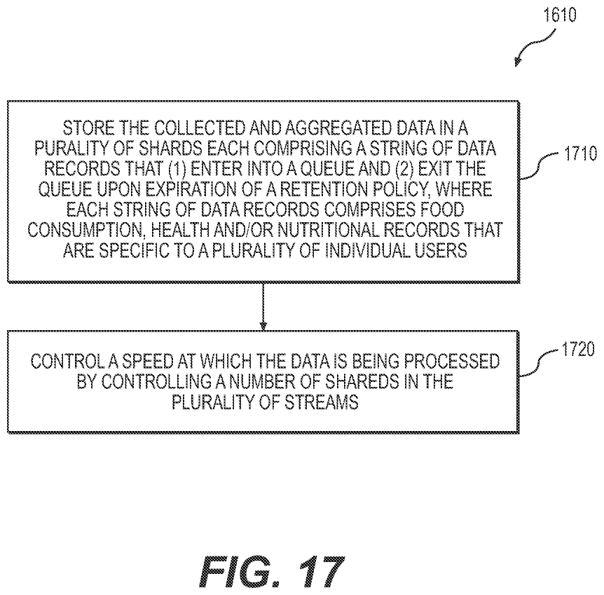

FIG. 17 is a flowchart that illustrates a method 1610 for storing collected and aggregated data from a plurality of different sources in a plurality of streams in accordance with the disclosed embodiments. The method 1610 begins at 1710 the retrieving module 230 stores collected and aggregated data from the plurality of different sources in the plurality of streams at the storage module 290, where the plurality of streams may comprise a plurality of shards, and each shard comprises a string of data records that (1) enter into a queue and (2) exit the queue upon expiration of the retention policy. The string of data records can include food consumption, health or nutritional records that are specific to a plurality of individual users. At 1720, the number of shards in the plurality of streams can be controlled to control the speed at which the data is processed.

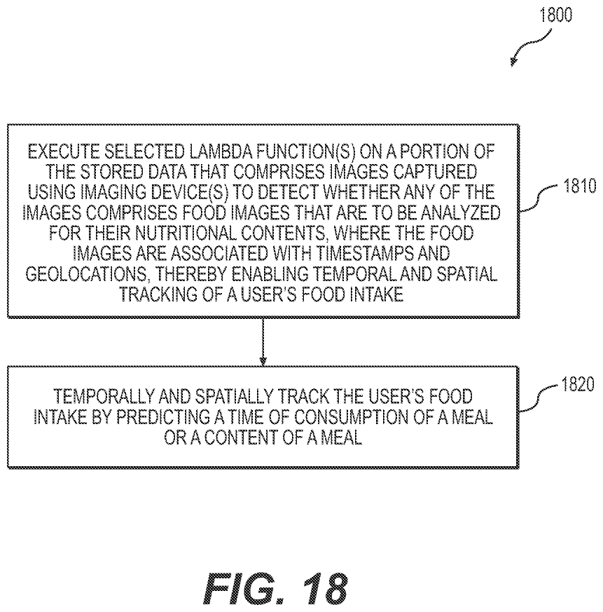

FIG. 18 is a flowchart that illustrates a method 1800 for analyzing images to determine their nutritional contents in accordance with the disclosed embodiments. Some of the stored collected and aggregated data can include images captured using one or more imaging devices. At 1810, one or more selected lambda function(s) can be executed on that portion of the stored data to detect whether any of the plurality of images comprises one or more food images that are to be analyzed for their nutritional contents. The food images are associated with timestamps and geolocations, thereby enabling, at 1820, temporal and spatial tracking of a user's food intake, for example, by predicting a time of consumption of a meal or a content of a meal.

While a computer-readable storage medium can be a single medium, the term “computer-readable storage medium” and the like should be taken to include a single medium or multiple media (e.g., a centralized or distributed database, and/or associated caches and servers) that store the one or more sets of instructions. The term “computer-readable storage medium” and the like shall also be taken to include any medium that is capable of storing, encoding or carrying a set of instructions for execution by the machine and that cause the machine to perform any one or more of the methodologies of the present invention. The term “computer-readable storage medium” and the like shall accordingly be taken to include, but not be limited to, solid-state memories, optical media, and magnetic media.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the scope of the present invention.

In the above description, numerous details are set forth. It will be apparent, however, to one of ordinary skill in the art having the benefit of this disclosure, that embodiments of the invention may be practiced without these specific details. In some instances, well-known structures and devices are shown in block diagram form, rather than in detail, in order to avoid obscuring the description.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “determining,” “identifying,” “adding,” “selecting,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

Embodiments of the invention also relate to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

The algorithms and displays presented herein are not inherently related to any particular computer or other apparatus. Various systems may be used with programs in accordance with the teachings herein, or it may prove convenient to construct a more specialized apparatus to perform the required method steps. The required structure for a variety of these systems will appear from the description provided herein. In addition, the present invention is not described with reference to any particular programming language. It will be appreciated that a variety of programming languages may be used to implement the teachings of the invention as described herein.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A computer-implemented data collection and processing method implemented using a serverless architecture for generating personalized dietary and health advice or recommendations via a hardware-based processing system, the method comprising:
collecting and aggregating data from a plurality of different sources in a storage module, wherein the data comprises different types or forms of data;
continuously processing each of the different types or forms of data in a manner that is agnostic of its source, by converting the different types or forms of data to a standardized structured format that is compatible with the health and nutrition platform;
analyzing the data that has been converted to the standardized structured format, using in part information from the health and nutrition platform; and
generating personalized dietary and health advice or recommendations for each of a plurality of individual users.

2. The method of claim 1, wherein the different types or forms of data comprise structured data and unstructured data.

3. The method of claim 1, wherein the data comprises food, health or nutritional data that is specific to a plurality of individual users.

4. The method of claim 1, wherein the standardized structured data is analyzed using one or more machine learning models comprising:
one or more artificial neural networks;
one or more regression models;
one or more decision tree models;
one or more support vector machines;
one or more Bayesian networks;
one or more probabilistic machine learning models;
one or more Gaussian processing models;
one or more Hidden Markov models; and
one or more deep learning networks.

5. The method of claim 1, wherein the data is collected and aggregated from the plurality of different sources through a plurality of Application Programming Interfaces (APIs).

6. The method of claim 5, further comprising: communicating with the plurality of APIs via a token module associated with one or more different entities, wherein the data from the plurality of APIs is collected and aggregated using a retrieving module, wherein the retrieving module is decoupled from and independent of the token module.

7. The method of claim 6, wherein the token module is configured to refresh existing tokens and provide notification updates about token changes, and wherein each time a new token is generated, the new token is separately duplicated in the retrieving module, in addition to being stored in the token module.

8. The method of claim 1, wherein the storage module is configured to verify, check and remove duplicate data, reduce the data by consolidating selected types of data, and to persist the data in batches.

9. The method of claim 1, wherein the collecting and aggregating of the data comprises storing the data in a plurality of streams.

10. The method of claim 9, wherein the processing of the data further comprises executing lambda functions on the data stored in the plurality of streams, upon occurrence of different conditions.

11. The method of claim 10, wherein the lambda functions are executed only when the data is collected and stored in the plurality of streams, and wherein the executing of the lambda functions on the stored data is configured to channel and transfer each row of data to a relevant stream from the plurality of streams.

12. The method of claim 11, wherein the data is advanced along a data pipeline by cascading from one stream to another stream of the plurality of streams.

13. The method of claim 9, where each of the plurality of streams has a retention policy defining a time frame in which the data is stored in each stream.

14. The method of claim 13, wherein the plurality of streams comprises a plurality of shards, and each shard comprises a string of data records that (1) enter into a queue and (2) exit the queue upon expiration of the retention policy, wherein the string of data records comprises food consumption, health or nutritional records that are specific to a plurality of individual users.

15. The method of claim 14, further comprising: controlling a speed at which the data is being processed, by controlling a number of shards in the plurality of streams.

16. The method of claim 1, wherein the collecting and aggregating of the data from the plurality of different sources comprises (1) pulling data from a first set of sources that permit data to be pulled at predetermined time intervals using a task scheduler, and (2) receiving data that is pushed from a second set of sources, such that the data from a plurality of pull requests and push requests are streamed into a centralized location, wherein the pushing of the data from the second set of sources is preceded by one or more notifications associated with the data, and (3) pulling data associated with a corresponding notification if said data does not arrive with the corresponding notification.

17. The method of claim 1, wherein a portion of the stored data comprises a plurality of images captured using one or more imaging devices, wherein a selected lambda function is executed on the portion of the stored data, to detect whether any of the plurality of images comprises one or more food images that are to be analyzed for their nutritional contents.

18. The method of claim 17, wherein the one or more food images are associated with timestamps and geolocations, thereby enabling temporal and spatial tracking of a user's food intake, wherein the temporal and spatial tracking of the user's food intake comprises predicting a time of consumption of a meal or a content of a meal.

19. A data collection and processing system implemented using a serverless architecture for generating personalized dietary and health advice or recommendations via a hardware-based processing system, wherein the system comprises at least one hardware-based processor and memory, wherein the memory comprises processor-executable instructions encoded on a non-transient processor-readable media, wherein the processor-executable instructions, when executed by the processor, are configurable to cause:
collecting and aggregating data from a plurality of different sources in a storage module, wherein the data comprises different types or forms of data;
continuously processing each of the different types or forms of data in a manner that is agnostic of its source, by converting the different types or forms of data to a standardized structured format that is compatible with a health and nutrition platform;
analyzing the data that has been converted to the standardized structured format, using in part information from the health and nutrition platform; and
generating personalized dietary and health advice or recommendations for each of a plurality of individual users.

20. A serverless data collection and processing system for generating personalized dietary and health advice or recommendations, the system comprising:
a retrieving module, that when executed by a hardware-based processing system, is configurable to cause: collecting and aggregating data from a plurality of different sources in a storage module, wherein the data comprises different types or forms of data; and
a standardization module, that when executed by the hardware-based processing system, is configurable to cause: continuously processing each of the different types or forms of data in a manner that is agnostic of its source, by converting the different types or forms of data to a standardized structured format that is compatible with a health and nutrition platform; and
a platform having one or more machine learning models, that when executed by the hardware-based processing system, is configurable to cause: analyzing the data that has been converted to the standardized structured format, using in part information from the health and nutrition platform; and generating personalized dietary and health advice or recommendations for each of the plurality of individual users.
